(19) United States
(12) Patent Application Publication
(10) Pub. No.: US 2012/0277236 A1
(43) Pub. Date: Nov. 1, 2012

(54) METHOD FOR INHIBITING MMP-9 DIMERIZATION

(76) Inventors: Jian CAO, S. Setauket, NY (US); Antoine DUFOUR, Quebec City (CA)

(21) Appl. No.: 13/457,246

(22) Filed: Apr. 26, 2012

Related U.S. Application Data
(60) Provisional application No. 61/479,262, filed on Apr. 26, 2011.

(51) Int. Cl. C12N 9/99; A61P 35/00; A61K 31/53; A61K 31/505; A61K 31/4245

(52) U.S. Cl. 514/245; 435/184; 514/274; 514/364

(57) ABSTRACT

A method of inhibiting matrix metalloproteinase 9 (MMP-9) dimerization without substantially inhibiting the catalytic activity of MMP-9, comprising contacting the MMP-9 with a small molecule compound of the structure

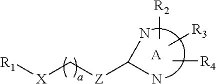

wherein
A is a ring structure which is substituted by R₂, R₃ and R₄;
X is present or absent and when present is NH, O, ester or N-monosubstituted amide;
Z is S, O, or N;
n is an integer from 0-5;
R₁ is alkyl, aryl, heteroalkyl, or heteroaryl, each with or without substitution; and
R₂, R₃, and R₄ is each present or absent, and is each independently H, alkyl, aryl, heteroalkyl, heteroaryl, each with or without substitution,
   or a pharmaceutically acceptable salt thereof.

This application claims the benefit of U.S. Provisional Application No. 61/479,262, filed Apr. 26, 2011, the contents of which are hereby incorporated by reference into this application.

The invention was made with government support under Grant number CA113553 awarded by the National Institutes of Health/National Cancer Institute. The government has certain rights in the invention.

Throughout this application, certain publications are referenced in parentheses. Full citations for these publications may be found immediately preceding the claims. The disclosures of these publications in their entireties are hereby incorporated by reference into this application in order to describe more fully the state of the art to which this invention relates.

### BACKGROUND OF THE INVENTION

Mortality in cancer is primarily due to failure to prevent metastasis. Much attention has been focused on targeting tumor growth; drug discovery targeting metastasis has lagged far behind. Thus, there is a pressing need for novel treatment strategies to prevent metastasis. Emerging evidence has emphasized the role of matrix metalloproteinases (MMPs) in early aspects of cancer dissemination (1-3). The demonstration that several MMPs display pro-tumor, as well as anti-tumor effects (4), highlights that more specific inhibitory drugs are required for clinical development.

MMPs have also been implicated in other disease entities, leading to the development of numerous drugs, which interfere with MMP enzymatic activity (5). Several classes of compounds, including peptidomimetics, tetracyclines and bisphosphonates, have been designed to bind and inhibit the catalytic activity of MMPs (6, 7). However, the catalytic domains of all MMPs share a highly conserved binding site and lack of specificity of these MMP inhibitors (MMPIs) has hindered their development as drugs. After the failure of broad-spectrum MMPIs in the treatment of cancer in phase III clinical trials, a re-evaluation of the biological roles of the MMPs has been undertaken (8).

A major conceptual advance in the development of novel MMPIs is to target less conserved, non-catalytic domains of the proteases to increase target specificity and selectivity. The critical importance of exosites of MMP's is highlighted by the fact that tissue inhibitor of metalloproteinase-1 (TIMP-1) and TIMP-2 can selectively bind to the hemopexin (PEX) domain of proMMP-9 and proMMP-2, respectively. In fact, exosites are crucial for the catalytic functions of most MMPs; enzyme lacking the PEX domain or the addition of an exogenous PEX domain greatly inhibits the proteolytic efficiency of the enzyme (9-11). Because the PEX domains of MMPs are not as highly conserved as the catalytic sites, the PEX domain is an alternative site that can inhibit the biological roles of MMPs with greater selectivity (12, 13). Novel therapeutics targeting MMP exosites are currently being evaluated with a focus to develop drugs with fewer side effects than previously developed broad-spectrum catalytic-site inhibitors (8, 14).

MMP-9 is linked to many pathological processes including cancer invasion, metastasis, and angiogenesis, as well as cardiovascular, neurologic and inflammatory diseases (2, 3, 15). Elevated levels of MMP-9 in tissue and blood are observed in these conditions. Active MMP-9 is an attractive target for cancer therapy development (16). The ability of MMP-9 to degrade collagen and laminin correlates with its ability to regulate cell migration, increase angiogenesis and affect tumor growth (15, 17). In addition to the effects of activated MMP-9 in degrading substrates and cleaving biologically relevant proteins, proMMP-9 induces cell migration independent of any proteolytic activity (12, 13, 17). Enhanced epithelial cell migration is linked to the formation of homodimers through the MMP-9 PEX domain, as well as heterodimers with other cell surface molecules (12, 13).

Described herein, is a method of inhibiting MMP-9 dimerization by selectively binding the PEX domain of MMP-9 with a series of small molecule compounds.

### SUMMARY OF THE INVENTION

This invention provides a method of inhibiting matrix metalloproteinase 9 (MMP-9) dimerization without substantially inhibiting the catalytic activity of MMP-9, comprising contacting the MMP-9 with a small molecule compound of the structure

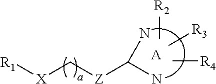

wherein
A is a ring structure which is substituted by R₂, R₃ and R₄;
X is present or absent and when present is NH, O, ester or N-monosubstituted amide;
Z is S, O, or N;
n is an integer from 0-5;
R₁ is alkyl, aryl, heteroalkyl, or heteroaryl, each with or without substitution; and
R₂, R₃, and R₄ is each present or absent, and is each independently H, alkyl, aryl, heteroalkyl, heteroaryl, each with or without substitution,

or a pharmaceutically acceptable salt thereof.

This invention provides a method for reducing one or more symptoms of disease in a mammal, comprising administering to the mammal a small molecule compound of the structure

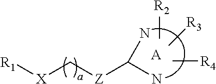

wherein
A is a ring structure which is substituted by R₂, R₃ and R₄;
X is present or absent and when present is NH, O, ester or N-monosubstituted amide;
Z is S, O, or N;
n is an integer from 0-5;
R₁ is alkyl, aryl, heteroalkyl, or heteroaryl, each with or without substitution; and
R₂, R₃, and R₄ is each present or absent, and is each independently H, alkyl, aryl, heteroalkyl, heteroaryl, each with or without substitution,

or a pharmaceutically acceptable salt thereof.

This invention provides a small molecule compound for use in inhibiting MMP-9 dimerization without substantially inhibiting the catalytic activity of MMP-9, the compound having the structure

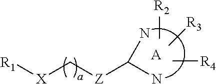

wherein
A is a ring structure which is substituted by R₂, R₃ and R₄;
X is present or absent and when present is NH, O, ester or N-monosubstituted amide;
Z is S, O, or N;
n is an integer from 0-5;
R₁ is alkyl, aryl, heteroalkyl, or heteroaryl, each with or without substitution; and

R₂, R₃, and R₄ is each present or absent, and is each independently H, alkyl, aryl, heteroalkyl, heteroaryl, each with or without substitution,

or a pharmaceutically acceptable salt thereof.

This invention provides a small molecule compound for use in reducing one or more symptoms of disease in a mammal, the compound having the structure

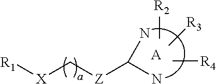

wherein
A is a ring structure which is substituted by R₂, R₃ and R₄;
X is present or absent and when present is NH, O, ester or N-monosubstituted amide;
Z is S, O, or N;
n is an integer from 0-5;
R₁ is alkyl, aryl, heteroalkyl, or heteroaryl, each with or without substitution; and
R₂, R₃, and R₄ is each present or absent, and is each independently H, alkyl, aryl, heteroalkyl, heteroaryl, each with or without substitution,

or a pharmaceutically acceptable salt thereof.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. Clinical relevance of MMP-9 in patients with breast cancer: MMP-9 expression is correlated with breast cancer recurrence and death. DNA microarray data mining of van de Vijver (25) and Stockholm (26) cohorts was performed using Kaplan-Meier survival analysis for correlation of MMP-9 expression with breast cancer survival rate (A & B) and recurrence (C). Levels of MMP-9 RNA were dichotomized at mean. n=cases.

FIG. 2. Identification of small molecular weight compounds bound to the MMP-9 PEX domain. A) Ribbon model of the PEX domain of MMP-9 (PDB code 1ITV, subunit A) with the compounds 1, 2, 4 and 5 docked. The sulfate ions are shown for references, but were not included as part of the docking receptor. B) The same docked structure as shown in (A) rotated 90° about the X-axis with a solvent-accessible surface on the protein. C) Compounds 1, 2, 4 and 5 overlaid in their docked conformations. Atom colors: Carbon (grey), oxygen (red), nitrogen (blue), sulfur (yellow), and fluorine (green). The images and the solvent-accessible surface of MMP-9 PEX monomer were generated in UCSF Chimera (20). D) Structures of the best five docked molecules.

FIG. 3. Inhibition of migration of cells expressing MMP-9 by the selected compounds. A-E) Cell migration assay: COS-1 cells transfected with GFP cDNA or MMP-9 cDNA were incubated with the five compounds at different concentration for 30 minutes before being subjected to a Transwell chamber migration assay for an additional 6 h. Each concentration was assayed in triplicate and the experiments were repeated three times. *P<0.05. F) Cell cytotoxic assay: COS-1 cells were incubated with the five compounds (100 μM) for 24 hours followed by a cell viability assay. DMEM media alone and media containing thapsigargin (1 μM) were included as negative and positive controls, respectively. **P<0.01. G-H) Measurement of lethal dose of 50% (LD₅₀): COS-1 cells were treated with varying doses of compound 1 (G) or compound 2 (H) for 24 hours followed by a cell viability assay. Curve fitting was established and the IC₅₀ values were calculated using GraphPad software.

FIG. 4. TLM Specificity and dose-dependent inhibition of MMP-9 induced cell migration and invasion by the selected compounds. A) Specificity of compounds for inhibition of MMP-9-induced cell migration: COS-1 cells transfected with cDNAs encoding MMP-2, MMP-9 or MT1-MMP were incubated with compound 1 (100 μM) or 2 (100 μM) for 30 minutes followed by a Transwell chamber migration assay. Each data point was performed in triplicate and the experiments were repeated three times. *P<0.05, as compared to DMSO-treated COS-1 cells transfected with MMP-9 cDNA. B-C) Dose-dependent inhibition of cancer cell migration by the selected compounds. Human fibrosarcoma HT-1080 cells and MDA-MB-435 cancer cells were incubated with 1% DMSO, or different concentrations of compound 1 or 2 for 30 minutes followed by a Transwell chamber migration assay. D-E) Reduction of HT-1080 cell invasion by the selected compounds. HT-1080 cells (1×104) were pre-treated with 1% DMSO, compound 1 (100 μM) or 2 (100 μM) for 30 minutes followed by dotting onto a 96-well plate with type 1 collagen. The cell-matrix was then covered by type 1 collagen gel with medium containing either a DMSO control or the selected compounds. Invading cells at the cell-collagen interface were microscopically counted after an 18-hour incubation.

FIG. 5. Specificity of small molecule inhibitors for the PEX domain of MMP-9. A) The selected compounds did not affect MMP-9 expression: HT-1080 cells were treated with 1% DMSO, compounds 1 or 2 (100 μM) for 18 hours. The cell lysate was examined by Western blotting (WB) using antibodies against MMP-9 and α/β tubulin, respectively. B) The selected compounds did not inhibit AMPA-activated MMP-9 proteolytic activity: APMA-activated MMP-9 was incubated with DMSO control or the selected compounds 1 and 2 (100 μM) for 3 hours at 37° C. followed by incubating with fluorogenic substrate peptide for 30 min at room temperature. Negative controls included untreated proMMP-9 and compounds only. Proteolytic activity was monitored using a fluorescence plate reader. C-D) Compound 2 binds selectively to the PEX domain of MMP-9: The λₘₐₓ of tryptophan fluorescence emission (excitation at 280 nm) was monitored upon titration with compound 2 or buffer only as a control with (C) purified recombinant MMP-9 (50 nM) and (D) MMP-9/MMP-2_{PEX} chimera. The data shown are the average of three independent replicates (standard error bars). The data were fit to equation (1) to obtain the dissociation constant (K_{d}) for compound 2 and MMP-9. E) Compound 2 interferes with MMP-9 homodimerization. COS-1 cells transfected with MMP-9-Myc and MMP-9-HA in the presence or absence of compounds 2 and 4 (100 μM) were pulled down with anti-HA antibody followed by Western blotting with anti-Myc antibody. The aliquots of total cell lysates serving as input were examined by Western blotting using anti-Myc antibodies. Reciprocal co-immunoprecipitation was also performed using anti-Myc antibody for pull down and anti-HA antibody for Western blotting. F) Compound 2 decreased MMP-9-mediated ERK1/2 activation: COS-1 cells transiently transfected with vector control and MMP-9 cDNAs were serum-starved for 18 hours in the presence or absence of compounds 2 and 4 (100 μM) followed by Western blotting using anti-pERK1/2 and total ERK1/2 antibodies.

FIG. 6. Inhibition of cell proliferation by compound 2. The effect of compound 2 on cell proliferation was examined in MMP-9 cDNA transfected COS-1 cells (A) or cancer cells expressing endogenous MMP-9 (HT-1080 and MDA-MB-435) (C & D), as well as GFP cDNA transfected COS-1 cells (control) (B) in the presence or absence of the compounds 2 and 4 (10 μM) for 9 days. *P<0.05, as compared to 1% DMSO or inactive compound 4.

FIG. 7: Retarded tumor growth and metastasis by compound 2 in mice bearing MDA-MB-435/GFP tumor xenografts. A-B) Effect of compound 2 on tumor growth: Mice bearing MDA-MB-435/GFP cells were administered compounds or vehicle control at 20 mg/kg (6 days per week). **P<0.01. Arrows indicate tumor mass. C-E) Inhibition of metastasis by compound 2: Lung sections ˜3 mm thick were examined by fluorescent microscopy. Incidence of metastasis was determined (C-D) and the average area of tumor in the lungs was determined using ImageJ software (E). **P<0.01.

FIG. 8: Assessment of compound 2 and 4 on MT1-MMP-mediated cell invasion: HeLa cells stably expressing GFP control or MT1-MMP-GFP (MT1-GFP) chimeric cDNAs were examined by the 3D invasion assay (23) in the presence or absence of compound 2 and 4 for 18 hours at 37° C. The cells were fixed and photographed (A). The invaded cells were microscopically counted (B). No inhibitory effect of compound 2 or 4 on MT1-MMP-mediated cancer cell invasion. ***P<0.001.

FIG. 9: No effect of compound 2 on mouse macrophage cell migration. A) Mouse RAW264.7 macrophage-like cells produce endogenous MMP-2 and -9: The conditioned medium from RAW264.7 cells in the presence or absence of the compounds as indicated was examined by gelatin zymography. No notable difference of MMP-2 or −9 expression was observed in compound treated cells. B) No inhibitory effect of compound 2 on RAW264.7 cell migration: RAW264.7 cells pre-treated with compounds as indicated for 30 min followed by a Transwell chamber migration assay for 16 hours. Each sample was triplicated and the experiment was repeated for two times.

FIG. 10: Homology analysis of the PEX and catalytic domains of MMP-9 with other MMPs: The catalytic and hemopexin domains of human MMPs were retrieved from the NCBI protein database. Each MMP was analyzed against the corresponding domain of MMP-9 using the Blast2 Alignment Program (http://blast.ncbi.nlm.nih.gov/Blast.cgi). In this analysis, scoring parameters include: 1) matrix non-default value: Blosum62; 2) Gap Costs: (Existence:11 Extension:1); 3) compositional adjustment: conditional compositional score matrix adjustment. Percent identity indicates the exact match between the sequences, and percent similarity shows the sum of both identical and similar matches between two sequences.

FIG. 11: Ranking of the top five selected compounds from the 100-compound ZINC test set.

### DETAILED DESCRIPTION OF THE INVENTION

This invention provides a method of inhibiting matrix metalloproteinase 9 (MMP-9) dimerization without substantially inhibiting the catalytic activity of MMP-9, comprising contacting the MMP-9 with a small molecule compound of the structure

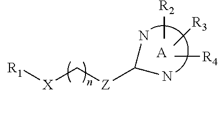

wherein
A is a ring structure which is substituted by R₂, R₃ and R₄;
X is present or absent and when present is NH, O, ester or N-monosubstituted amide;
Z is S, O, or N;
n is an integer from 0-5;
R₁ is alkyl, aryl, heteroalkyl, or heteroaryl, each with or without substitution; and

R₂, R₃, and R₄ is each present or absent, and is each independently H, alkyl, aryl, heteroalkyl, heteroaryl, each with or without substitution,

or a pharmaceutically acceptable salt thereof.

In some embodiments, the method includes the compound wherein in the compound the ring structure A is an aromatic or non-aromatic monocycle, bicycle, mono-heterocycle, or bi-heterocycle, each with or without substitution,

or a pharmaceutically acceptable salt thereof.

In some embodiments, the method includes the compound wherein R₁ is an aromatic or non-aromatic monocycle, bicycle, mono-heterocycle, or bi-heterocycle, each with or without substitution,

or a pharmaceutically acceptable salt thereof.

In some embodiments, the method includes the compound wherein at least one of ring structure A or R₁ is phenyl, pyrimidine, pyridine, imidazole, triazine, triazole, pyrimidinone, triazolotriazine, or benzimidazole, each with or without substitution,

or a pharmaceutically acceptable salt thereof.

In some embodiments, the method includes the compound wherein the ring structure A is phenyl, pyrimidine, pyridine, imidazole, triazine, triazole, pyrimidinone, or triazolotriazine, each with or without substitution.

or a pharmaceutically acceptable salt thereof.

In some embodiments, the method includes the compound wherein in the compound the ring structure A is

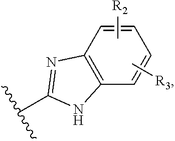

or a pharmaceutically acceptable salt thereof.

In some embodiments, the method includes the compound wherein in the compound the ring structure A is

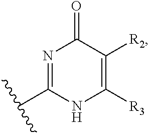

or a pharmaceutically acceptable salt thereof.

In some embodiments, the method includes the compound wherein in the compound the ring structure A is

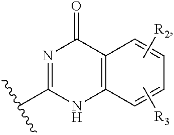

or a pharmaceutically acceptable salt thereof.

In some embodiments, the method includes the compound wherein in the compound the ring structure A is

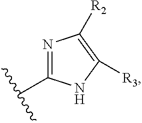

or a pharmaceutically acceptable salt thereof.

In some embodiments, the method includes the compound wherein in the compound the ring structure A is

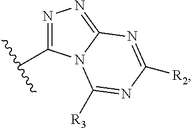

or a pharmaceutically acceptable salt thereof.

In some embodiments, the method includes the compound wherein R₄ is absent, and R₂ and R₃ are each independently H, alkyl, heteroalkyl, aryl or heteroaryl, each with or without substitution.

or a pharmaceutically acceptable salt thereof.

In some embodiments, the method includes the compound wherein X is present and X is

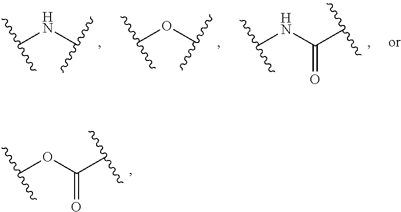

or a pharmaceutically acceptable salt thereof.

In some embodiments, the method includes the compound wherein n=1, 2, or 3;

X is present and X is

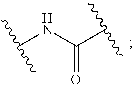

and

R₁ is unsubstituted phenyl, monosubstituted phenyl, disubstituted phenyl or trisubstituted phenyl,

or a pharmaceutically acceptable salt thereof.

In some embodiments, the method includes the compound wherein R₁ is

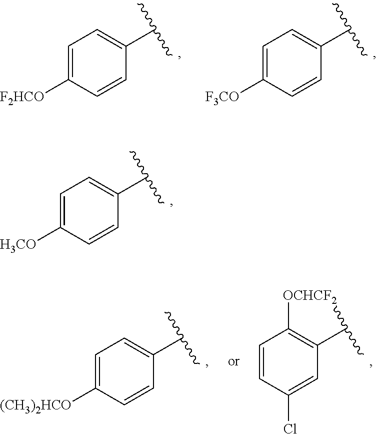

or a pharmaceutically acceptable salt thereof.

In some embodiments, the method includes the compound wherein X is absent; n=1, 2, or 3; and

R₁ is unsubstituted phenyl, monosubstituted phenyl, disubstituted phenyl, trisubstituted phenyl, or pyrimidinone, with or without substitution, fused or unfused.

or a pharmaceutically acceptable salt thereof.

In some embodiments, the method includes the compound wherein R₁ is

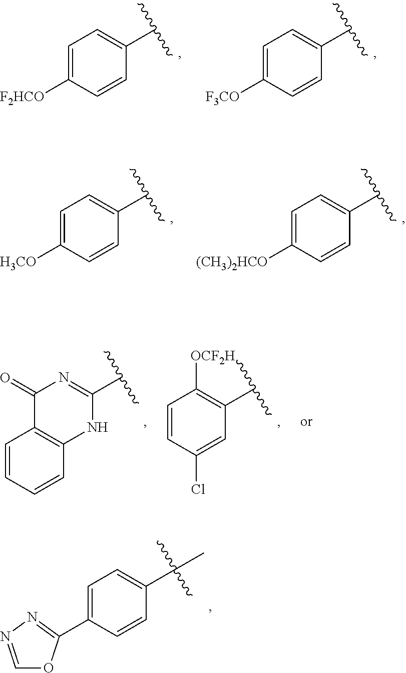

or a pharmaceutically acceptable salt thereof.

In some embodiments, the method includes the compound wherein R₁ is

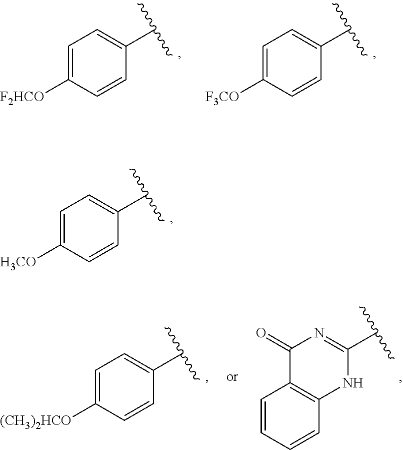

or a pharmaceutically acceptable salt thereof.

In some embodiments, the method includes the compound wherein the structure is

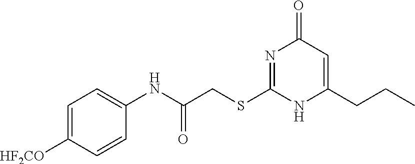

or a pharmaceutically acceptable salt thereof.

In some embodiments, the method includes the compound wherein the structure is

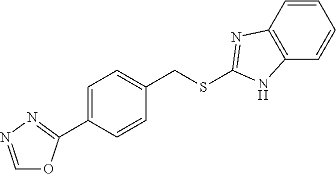

or a pharmaceutically acceptable salt thereof.

In some embodiments, the method includes the compound wherein the structure is

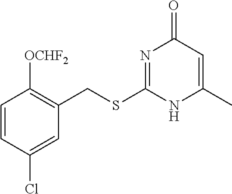

or a pharmaceutically acceptable salt thereof.

In some embodiments, the method includes the compound wherein the structure is

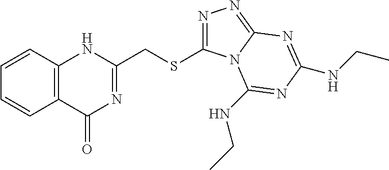

or a pharmaceutically acceptable salt thereof.

In some embodiments, the invention provides a method for reducing one or more symptoms of disease in a mammal, comprising administering to the mammal a small molecule compound of the structure

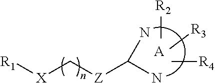

wherein
A is a ring structure which is substituted by R₂, R₃ and R₄;
X is present or absent and when present is NH, O, ester or N-monosubstituted amide;
Z is S, O, or N;
n is an integer from 0-5;
R₁ is alkyl, aryl, heteroalkyl, or heteroaryl, each with or without substitution; and
R₂, R₃, and R₄ is each present or absent, and is each independently H, alkyl, aryl, heteroalkyl, heteroaryl, each with or without substitution,

or a pharmaceutically acceptable salt thereof.

In some embodiments, the invention provides a method includes the compound wherein the disease is cancer.

In some embodiments, the invention provides a method of reducing one or more symptoms of cancer in a mammal.

In some embodiments, the method includes the compound that inhibits cancer cell metastasis.

In some embodiments, the method includes the compound that inhibits cancer cell proliferation.

In some embodiments, the method includes the compound that inhibits cancer cell migration.

In some embodiments, the method includes the compound that inhibits cell metastasis in breast cancer cells.

In some embodiments, the method includes the compound that inhibits cell migration in breast cancer cells.

In some embodiments, the method includes the compound that inhibits cell proliferation in breast cancer cells.

In some embodiments, a small molecule compound for use in inhibiting MMP-9 dimerization without substantially inhibiting the catalytic activity of MMP-9, the compound having the structure

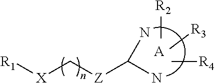

wherein
A is a ring structure which is substituted by R₂, R₃ and R₄;
X is present or absent and when present is NH, O, ester or N-monosubstituted amide;
Z is S, O, or N;
n is an integer from 0-5;
R₁ is alkyl, aryl, heteroalkyl, or heteroaryl, each with or without substitution; and
R₂, R₃, and R₄ is each present or absent, and is each independently H, alkyl, aryl, heteroalkyl, heteroaryl, each with or without substitution,

or a pharmaceutically acceptable salt thereof.

In some embodiments, a small molecule compound for use in reducing one or more symptoms of disease in a mammal, the compound having the structure

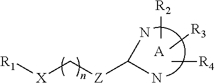

wherein
A is a ring structure which is substituted by H2, R₃ and R₄;
X is present or absent and when present is NH, O, ester or N-monosubstituted amide;
Z is S, O, or N;
n is an integer from 0-5;
R₁ is alkyl, aryl, heteroalkyl, or heteroaryl, each with or without substitution; and
R₂, R₃, and R₄ is each present or absent, and is each independently H, alkyl, aryl, heteroalkyl, heteroaryl, each with or without substitution,

or a pharmaceutically acceptable salt thereof.

As used herein, “catalytic activity” is the ability of an enzyme to catalyze a chemical reaction when a specific substrate is bound to the catalytic binding site.

As used herein, “homodimerization” refers to the oligomerization between two polypeptides having the same amino acid sequence.

As used herein, “matrix metalloproteinases” refers to a family of nine or more highly homologous Zn(++)-endopeptidases that collectively cleave most if not all of the constituents of the extracellular matrix, and act on pro-inflammatory cytokines, chemokines and other proteins to regulate varied aspects of inflammation and immunity.

As used herein, “matrix metalloproteinase-9” and “MMP-9” refer to a distinct matrix metalloproteinase that contains a signal peptide, N-terminal propeptide, catalytic domain that contains three fibronectin type II repeats, hinge region, and a C-terminal “hemopexin domain,” also referred to as “PEX domain” and “MMP-9-PEX domain.” See Matrix Metalloproteinases and TIMPs, J. Frederick Woessner, Hideaki Nagase, (Oxford University Press) 2nd Edition (2000) and Matrix Metalloproteinase Inhibitors in Cancer Therapy, Neil J. Clendeninn, Krzysztof Appelt, (Humana Press) 1st Edition (2011) and references therein.

In some embodiments, the invention provides a method of reducing one or more symptoms of any disease that involves MMP-9-induced cell migration.

In some embodiments, the disease is exemplified by cancer, cancer metastasis, systemic lupus erythematosus (SLE), Sjogren's syndrome (SS), systemic sclerosis (SS), polymyositis, rheumatoid arthritis (RA), multiple sclerosis (MS), atherosclerosis, cerebral ischemia, abdominal aortic aneurysm (AAA), myocardial infarction (MI), cerebral amyloid angiopathy (CAA), angiogenesis, inflammation, ectopic eczema, and contact eczema.

In some embodiments, the invention provides a method of reducing one or more symptoms of any disease that involves carcinomas including but not limited to lung cancer, breast cancer, prostate cancer, cervical cancer, pancreatic cancer, colon cancer, ovarian cancer; stomach cancer, esophagus cancer, mouth cancer, tongue cancer, gum cancer, skin cancer (e.g., melanoma, basal cell carcinoma, Kaposi's sarcoma, etc.), muscle cancer, heart cancer, liver cancer, bronchial cancer, cartilage cancer, bone cancer, testis cancer, kidney cancer, endometrium cancer, uterus cancer, bladder cancer, bone marrow cancer, lymphoma cancer, spleen cancer, thymus cancer, thyroid cancer, brain cancer, neuron cancer, mesothelioma, gall bladder cancer, ocular cancer (e.g., cancer of the cornea, cancer of uvea, cancer of the choroids, cancer of the macula, vitreous humor cancer, etc.), joint cancer (such as synovium cancer), glioblastoma, lymphoma, and leukemia. Malignant neoplasms are further exemplified by sarcomas (such as osteosarcoma and Kaposi's sarcoma).

In some embodiments, the invention provides a method of reducing one or more symptoms of any cancers that have been found to have upregulated MMP-9, including but not limited to breast, brain and CNS, gastrointestinal, head and neck, kidney, lung, lymphoma, melanoma, ovarian cancers, sarcoma, neuroblastoma, and lymphoblastic cancer.

In some embodiments, the cancer cell is a metastatic cancer cell, including but not limited to cancer cell lines MCF-7, MDA-MB-231, MDA-435, HT-1080, LNCaP, DU145, PC3, TK4, C-1H, C-26, Co-3, HT-29, KM12SM, and 253F B-V.

In some embodiments, the invention provides a method of reducing one or more symptoms of a disease which comprises of increased cell migration in the presence of MMP-9 compared to in the absence of MMP-9.

Except where otherwise specified, when the structure of a compound of this invention includes an asymmetric carbon atom, it is understood that the compound occurs as a racemate, racemic mixture, and isolated single enantiomer. All such isomeric forms of these compounds are expressly included in this invention. Except where otherwise specified, each stereogenic carbon may be of the R or S configuration. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis, such as those described in “Enantiomers, Racemates and Resolutions” by J. Jacques, A. Collet and S. Wilen, Pub. John Wiley S Sons, NY, 1981. For example, the resolution may be carried out by preparative chromatography on a chiral column.

The subject invention is also intended to include all isotopes of atoms occurring on the compounds disclosed herein. Isotopes include those atoms having the same atomic number but different mass numbers. By way of general example and without limitation, isotopes of hydrogen include tritium and deuterium. Isotopes of carbon include C-13 and C-14.

It will be noted that any notation of a carbon in structures throughout this application, when used without further notation, are intended to represent all isotopes of carbon, such as ¹²C, ¹³C, or ¹⁴C. Furthermore, any compounds containing ¹³C or ¹⁴C may specifically have the structure of any of the compounds disclosed herein.

It will also be noted that any notation of a hydrogen in structures throughout this application, when used without further notation, are intended to represent all isotopes of hydrogen, such as ¹H, ²H, or ³H. Furthermore, any compounds containing ²H or ³H may specifically have the structure of any of the compounds disclosed herein.

Isotopically-labeled compounds can generally be prepared by conventional techniques known to those skilled in the art using appropriate isotopically-labeled reagents in place of the non-labeled reagents employed.

The term “substitution”, “substituted” and “substituent” refers to a functional group as described above in which one or more bonds to a hydrogen atom contained therein are replaced by a bond to non-hydrogen or non-carbon atoms, provided that normal valencies are maintained and that the substitution results in a stable compound. Substituted groups also include groups in which one or more bonds to a carbon(s) or hydrogen(s) atom are replaced by one or more bonds, including double or triple bonds, to a heteroatom. Examples of substituent groups include the functional groups described above, and halogens (i.e., F, Cl, Br, and I); alkyl groups, such as methyl, ethyl, n-propyl, isopropryl, n-butyl, tert-butyl, and trifluoromethyl; hydroxyl; alkoxy groups, such as methoxy, ethoxy, n-propoxy, and isopropoxy; aryloxy groups, such as phenoxy; arylalkyloxy, such as benzyloxy (phenylmethoxy) and p-trifluoromethylbenzyloxy(4-trifluoromethylphenylmethoxy); heteroaryloxy groups; sulfonyl groups, such as trifluoromethanesulfonyl, methanesulfonyl, and p-toluenesulfonyl; nitro, nitrosyl; mercapto; sulfanyl groups, such as methylsulfanyl, ethylsulfanyl and propylsulfanyl; cyano; amino groups, such as amino, methylamino, dimethylamino, ethylamino, and diethylamino; and carboxyl. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

In the compounds used in the method of the present invention, the substituents may be substituted or unsubstituted, unless specifically defined otherwise.

In the compounds used in the method of the present invention, alkyl, heteroalkyl, monocycle, bicycle, aryl, heteroaryl and heterocycle groups can be further substituted by replacing one or more hydrogen atoms with alternative non-hydrogen groups. These include, but are not limited to, halo, hydroxy, mercapto, amino, carboxy, cyano and carbamoyl.

It is understood that substituents and substitution patterns on the compounds used in the method of the present invention can be selected by one of ordinary skill in the art to provide compounds that are chemically stable and that can be readily synthesized by techniques known in the art from readily available starting materials. If a substituent is itself substituted with more than one group, it is understood that these multiple groups may be on the same carbon or on different carbons, so long as a stable structure results.

In choosing the compounds used in the method of the present invention, one of ordinary skill in the art will recognize that the various substituents, i.e. R₁, R₂, etc. are to be chosen in conformity with well-known principles of chemical structure connectivity.

As used herein, “alkyl” includes cyclic, branched and straight-chain saturated aliphatic hydrocarbons, and unless otherwise specified contains one to ten carbons. Examples include, but are not limited to, methyl, ethyl, n-propyl, isopropyl, n-butyl, t-butyl, pentyl, hexyl, and octyl. Alkyl groups can be unsubstituted or substituted with one or more substituents, including but not limited to halogen, alkoxy, alkylthio, trifluoromethyl, difluoromethyl, methoxy, and hydroxyl.

As used herein, “heteroalkyl” includes both branched and straight-chain saturated aliphatic hydrocarbon groups having the specified number of carbon atoms and at least 1 heteroatom within the chain or branch.

As used herein, “monocycle” includes any stable polycyclic carbon ring of up to 10 atoms and may be unsubstituted or substituted. Examples of such non-aromatic monocycle elements include but are not limited to: cyclobutyl, cyclopentyl, cyclohexyl, and cycloheptyl. Examples of such aromatic monocycle elements include but are not limited to: phenyl.

As used herein, “bicycle” includes any stable polycyclic carbon ring of up to 10 atoms that is fused to a polycyclic carbon ring of up to 10 atoms with each ring being independently unsubstituted or substituted. Examples of such non-aromatic bicycle elements include but are not limited to: decahydronaphthalene. Examples of such aromatic bicycle elements include but are not limited to: naphthalene.

As used herein, “aryl” is intended to mean any stable monocyclic, bicyclic or polycyclic carbon ring of up to 10 atoms in each ring, wherein at least one ring is aromatic, and may be unsubstituted or substituted. Examples of such aryl elements include but are not limited to: phenyl, p-toluenyl (4-methylphenyl), naphthyl, tetrahydro-naphthyl, indanyl, phenanthryl, anthryl or acenaphthyl. In cases where the aryl substituent is bicyclic and one ring is non-aromatic, it is understood that attachment is via the aromatic ring.

The term “heteroaryl” or “heterocycle”, as used herein, represents a stable monocyclic, bicyclic or polycyclic ring of up to 10 atoms in each ring, wherein at least one ring is aromatic and contains from 1 to 4 heteroatoms selected from the group consisting of O, N and S. Bicyclic aromatic heteroaryl groups include but are not limited to phenyl, pyridine, pyrimidine or pyridizine rings that are (a) fused to a 6-membered aromatic (unsaturated) heterocyclic ring having one nitrogen atom; (b) fused to a 5- or 6-membered aromatic (unsaturated) heterocyclic ring having two nitrogen atoms; (c) fused to a 5-membered aromatic (unsaturated) heterocyclic ring having one nitrogen atom together with either one oxygen or one sulfur atom; or (d) fused to a 5-membered aromatic (unsaturated) heterocyclic ring having one heteroatom selected from O, N or S. Heteroaryl groups within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, azetidinyl, aziridinyl, 1,4-dioxanyl, hexahydroazepinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, acridinyl, carbazolyl, cinnolinyl, quinoxalinyl, pyrrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetra-hydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

The term “ester” is intended to a mean an organic compound containing the R—O—CO—R′ group.

The term “monosubstituted amide” is intended to a mean an organic compound containing the R—CO—NH—R′ group.

The term “phenyl” is intended to mean an aromatic six membered ring containing six carbons.

The term “biphenyl” is intended to mean an aryl comprising two benzene rings linked together, and any substituted derivative thereof.

The term “triazole” is intended to mean a heteroaryl having a five-membered ring containing two carbon atoms and three nitrogen atoms, and any substituted derivative thereof.

The term “pyridine” is intended to mean a heteroaryl having a six-membered ring containing 5 carbon atoms and 1 nitrogen atom, and any substituted derivative thereof.

The term “pyrimidine” is intended to mean a heteroaryl having a six-membered ring containing 4 carbon atoms and 2 nitrogen atoms, and any substituted derivative thereof.

The term “triazine” is intended to mean a heteroaryl having a six-membered ring containing 3 carbon atoms and 3 nitrogen atoms, and any substituted derivative thereof.

The term “pyrimidinone” is intended to mean a heteroaryl having a six-membered ring containing 4 carbon atoms and 2 nitrogen atoms, with one hydroxyl group directly attached to one of the carbons in the ring structure adjacent to a nitrogen and any substituted derivative thereof.

The term “triazolotriazine” is intended to mean a heteroaryl having a five-membered ring fused to a six membered ring with a total of 4 carbon atoms and 5 nitrogen atoms which can be shared by either ring, with the five-membered ring containing two carbon atoms and three nitrogen atoms and the six-membered ring containing three carbon atoms and three nitrogen atoms and any substituted derivative thereof.

The term “benzimidazole” is intended to mean a heteroaryl having a five-membered ring fused to a phenyl ring with the five-membered ring containing 2 nitrogen atoms directly attached to the phenyl ring.

The term “phenyloxadiazol” is intended to mean a heteroaryl having a phenyl ring directly linked to a five-membered ring containing 2 nitrogen atoms, 1 oxygen atom and 2 carbon atoms.

The compounds used in the method of the present invention may be prepared by techniques well know in organic synthesis and familiar to a practitioner ordinarily skilled in the art. However, these may not be the only means by which to synthesize or obtain the desired compounds.

The compounds used in the method of the present invention may be prepared by techniques described in Vogel's Textbook of Practical Organic Chemistry, A. I. Vogel, A. R. Tatchell, B. S. Furnis, A. J. Hannaford, P. W. G. Smith, (Prentice Hall) 5^{th} Edition (1996), March's Advanced Organic Chemistry: Reactions, Mechanisms, and Structure, Michael B. Smith, Jerry March, (Wiley-Interscience) 5^{th} Edition (2007), and references therein, which are incorporated by reference herein. However, these may not be the only means by which to synthesize or obtain the desired compounds.

The compounds used in the method of the present invention may be purchased from a variety of chemical suppliers including custom and contract synthesis organizations, including Enamine Ltd (23 Alexandra Matrosova Street, Kiev, 01103, Ukraine), Aurora Fine Chemicals (7929 Silverton Avenue, Suite 609, San Diego, Calif., 92126, USA), and Interchim Inc. (1536 West 25^{th} Street, Suite 452 San Pedro, Calif., 90732 USA). However, these may not be the only means by which to synthesize or obtain the desired compounds.

The various R groups attached to the aromatic rings of the compounds disclosed herein may be added to the rings by standard procedures, for example those set forth in Advanced Organic Chemistry Part B: Reaction and Synthesis, Francis Carey and Richard Sundberg, (Springer) 5th ed. Edition. (2007), the content of which is hereby incorporated by reference.

Another aspect of the invention comprises a compound used in the method of the present invention as a pharmaceutical composition.

As used herein, the term “pharmaceutically active agent” means any substance or compound suitable for administration to a subject and furnishes biological activity or other direct effect in the treatment, cure, mitigation, diagnosis, or prevention of disease, or affects the structure or any function of the subject. Pharmaceutically active agents include, but are not limited to, substances and compounds described in the Physicians' Desk Reference (PDR Network, LLC; 64th edition; Nov. 15, 2009) and “Approved Drug Products with Therapeutic Equivalence Evaluations” (U.S. Department Of Health And Human Services, 30^{th} edition, 2010), which are hereby incorporated by reference. Pharmaceutically active agents which have pendant carboxylic acid groups may be modified in accordance with the present invention using standard esterification reactions and methods readily available and known to those having ordinary skill in the art of chemical synthesis. Where a pharmaceutically active agent does not possess a carboxylic acid group, the ordinarily skilled artisan will be able to design and incorporate a carboxylic acid group into the pharmaceutically active agent where esterification may subsequently be carried out so long as the modification does not interfere with the pharmaceutically active agent's biological activity or effect.

The compounds used in the method of the present invention may be in a salt form. As used herein, a “salt” is a salt of the instant compounds which has been modified by making acid or base salts of the compounds. In the case of compounds used to treat an infection or disease caused by a pathogen, the salt is pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium. The term “pharmaceutically acceptable salt” in this respect, refers to the relatively non-toxic, inorganic and organic acid or base addition salts of compounds of the present invention. These salts can be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge at al. (1977) “Pharmaceutical Salts”, J. Pharm. Sci. 66:1-19).

As used herein, “treating” means preventing, slowing, halting, or reversing the progression of a disease or infection. Treating may also mean improving one or more symptoms of a disease or infection.

The compounds used in the method of the present invention may be administered in various forms, including those detailed herein. The treatment with the compound may be a component of a combination therapy or an adjunct therapy, i.e. the subject or patient in need of the drug is treated or given another drug for the disease in conjunction with one or more of the instant compounds. This combination therapy can be sequential therapy where the patient is treated first with one drug and then the other or the two drugs are given simultaneously. These can be administered independently by the same route or by two or more different routes of administration depending on the dosage forms employed.

As used herein, a “pharmaceutically acceptable carrier” is a pharmaceutically acceptable solvent, suspending agent or vehicle, for delivering the instant compounds to the animal or human. The carrier may be liquid or solid and is selected with the planned manner of administration in mind. Liposomes are also a pharmaceutically acceptable carrier.

The dosage of the compounds administered in treatment will vary depending upon factors such as the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the compounds used in the method of the present invention may comprise a single compound or mixtures thereof with additional antibacterial agents. The compounds can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by injection, topical application, or other methods, into or onto a site of infection, all using dosage forms well known to those of ordinary skill in the pharmaceutical arts.

The compounds used in the method of the present invention can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous or direct injection or parenteral administration. The compounds can be administered alone or mixed with a pharmaceutically acceptable carrier. This carrier can be a solid or liquid, and the type of carrier is generally chosen based on the type of administration being used. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Oral dosage forms optionally contain flavorants and coloring agents. Parenteral and intravenous forms may also include minerals and other materials to make them compatible with the type of injection or delivery system chosen.

Techniques and compositions for making dosage forms useful in the present invention are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol. 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds used in the method of the present invention may also be administered in the form of liposome delivery systems, such as small unilamellar vesicles, large unilamallar vesicles, and multilamellar vesicles. Liposomes can be formed from a variety of phospholipids, such as cholesterol, stearylamine, or phosphatidylcholines. The compounds may be administered as components of tissue-targeted emulsions.

The compounds used in the method of the present invention may also be coupled to soluble polymers as targetable drug carriers or as a prodrug. Such polymers include polyvinylpyrrolidone, pyran copolymer, polyhydroxyipropylmethacrylamide-phenol, polyhydroxyethylasparta-midephenol, or polyethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Gelatin capsules may contain the active ingredient compounds and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field.

The compounds used in the method of the present invention may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen.

Parenteral and intravenous forms may also include minerals and other materials to make them compatible with the type of injection or delivery system chosen.

Each embodiment disclosed herein is contemplated as being applicable to each of the other disclosed embodiments. Thus, all combinations of the various elements described herein are within the scope of the invention.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

### Example 1

### Materials and Methods

Cell Culture, Reagents and Transfection

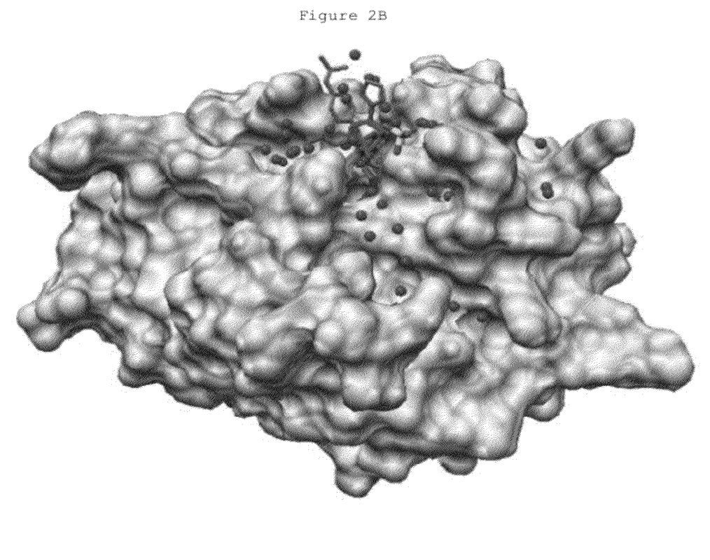

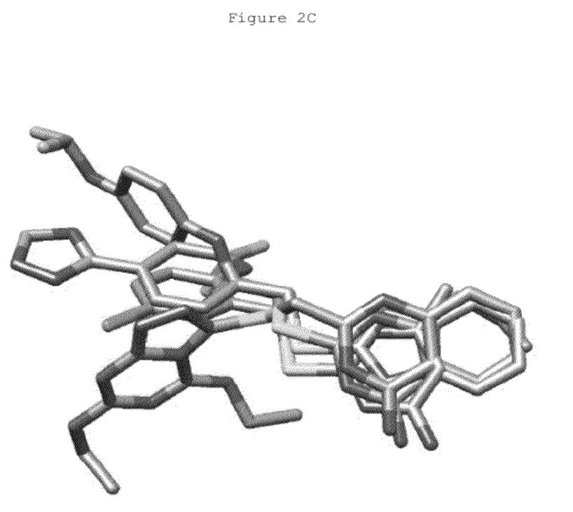

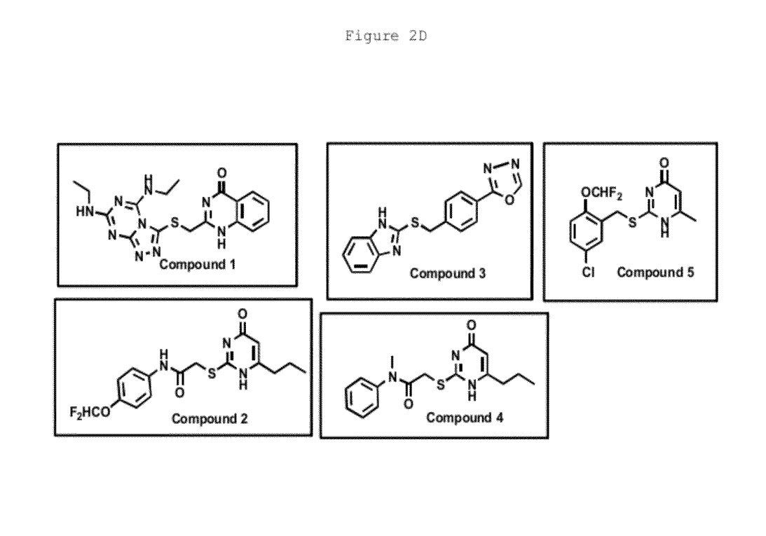

COS-1 monkey epithelial, human HT-1080, and MDA-MB-435 cancer cell lines, and murine macrophage-like RAW246.7 cell line were purchased from ATCC (Manassas, Va.) and were maintained in Dulbecco's modified Eagle's medium (Invitrogen) containing 10% fetal calf serum. Transfection of plasmid DNA (human) into cells was achieved using polyethylenimine (Polysciences) and the transfected cells were incubated for 48 h at 37° C. followed by biochemical and biological assays. Inhibitors were incubated with cells for 30 min prior to Transwell chamber migration assays. MMP-9 and MMP-9/MMP-2_{PEX} (13) proteins were purified from transfected cell-conditioned media by gelatin-Sepharose chromatography. Compounds 1-5 (FIG. 2D) were purchased from Enamine Ltd. (Kiev, Ukraine) and their purity was verified by LC/MS to be greater than 98% (Agilent 1100, Kinetex C18, 2.6 μm, 100 Å, 2.1×100 mm, solvent A: 10 mM NH₄OAc, pH 6.5/CH₃CN, 95:5, solvent 8: 10 mM NH₄OAc, pH 6.5/CH₃CN, 5:95, linear gradient: 5-95% B over 20 min at 0.4 mL/min, 30° C.). Anti-ERK1/2, anti-phospho ERK1/2 antibodies were purchased from Cell Signaling Technology (Davers, Mass.). Mac-P-L-G-L-Dpa-A-R-NH₂ fluorogenic peptide was obtained from R & D Systems (Minneapolis, Minn., USA).

DOCK 6.0 Calculations

Three-dimensional coordinates of the MMP-9 structure were obtained from the Protein Data Bank (PDB) (http://www.wwpdb.org) (18). The MMP-9 hemopexin structure (file 1ITV, http://dx.doi.org/10.2210/pdb1ITV/pdb) had a 1.95 Å resolution (19). Ions and water molecules were removed from the structure and hydrogens atoms were added. The structure was minimized to remove any steric strain that had been introduced and the partial atomic charges were calculated using a Gasteiger force field. All the residues and charges were visually inspected to ensure appropriateness and consistency.

DOCK 6.0 was used to calculate the protein/ligand binding energies. The calculations were performed starting from the X-ray crystal structure that was already minimized against experimental diffraction data. Energy contributions for individual atoms were extracted from the DOCK energy output. The grid size was set to 100×100×100 points with a grid spacing of 0.3 Å centered on the middle of monomer A of the PEX domain. The grid box included the entire subunit domain and provided enough space for ligand translational and rotational walk. Step sizes of 1.0 Å for translation and 50° for rotation were chosen and the cluster RMSD threshold was set at 2.0 Å. The number of ligands per cluster was set at 100. After generation and selection of spheres, the grid step was applied to 100 commercially available compounds from the ZINC 2007 database. Hits were ranked according to cluster size, grid score energy, van der Waals energy and electrostatic energy. Molecular graphic images were produced using the UCSF Chimera package from the Resource for Biocomputing, Visualization and Informatics at the University of California, San Francisco (20).

Fluorogenic Assay of Enzyme Activity

Fluorogenic peptide substrate (50 μM) (21) was incubated with the compounds either in the presence or absence of latent MMP-9 and APMA-activated MMP-9 for 30 min at 25° C. before detection. Fluorescence emission at 393 nm with excitation at 328 nm was measured in a fluorescent plate reader (SpectraMax M5, Molecular Devices).

Fluorescence Spectroscopy

Binding of compound 2 to MMP-9 was assayed by observing the change of tryptophan emission upon binding. Recombinant proteins were purified from COS-1 cells transfected with the appropriate cDNA using gelatin-Sepharose chromatography (22). Recombinant MMP-9 (50 nM) or MMP-9/MMP-2_{PEX} (50 nM) was added to a cuvette (1.0 mL final volume) containing buffer (50 mM Tris-HCl, 60 mM KCl and 0.05% Tween 20, pH 7.4). Compound 2 was prepared as a DMSO stock solution (50 mM stock solution) and aliquots added to the protein sample with stirring. Appropriate dilutions of the stock solution into buffer were made before titration. As a control for protein stability and loss, an analogous buffer solution was added to the protein. The protein sample was excited at 280 nm and emission scans were collected from 290 to 400 nm, using slit widths of 0.3 nm on a QM-4/200SE spectrofluorimeter with double excitation and emission monochromators. Three emission scans were collected and averaged at each concentration. The K_{d} was determined using the Prism software package (GraphPad V5) to fit the data to equation (1).

λₘₐₓ=(λₘₐₓ^{∞}*[2])/(K_{d}+[2]) (1)

in which λₘₐₓ is the wavelength at which maximal fluorescence of the protein was observed for a given concentration of inhibitor.

Cell Viability

Compound cytotoxicity was determined using the CellTiter-Glo™ Luminescent Cell Viability Assay generating luminescent signals based on quantification of ATP levels (Promega Corporation, Madison, Wis.). 2.5×10⁴ COS-1 cells were added to an opaque-walled 96-well plate and incubated for 18 hours with compounds 1-5 (100 μM). The plate was then equilibrated for 30 min at room temperature before adding the CellTiter-Glo® Reagent. The solution was mixed for 2 min to induce cell lysis. The plate was incubated for 10 min at room temperature before luminescence was recorded using a SpectraMax Microplate

Reader (Molecular Devices). LD₅₀'s of compounds 1 and 2 were measured in an analogous fashion with a range of linear doses (100 μM to 10 mM). The LD₅₀ was determined using the Prism software package (GraphPad V5) and fitting to equation (2).

ΔL=(ΔLₘₐₓ*[inhibitor])(LD₅₀+[inhibitor]) (2)

in which L=the measured luminescence.

Cell Proliferation

Cell proliferation was determined using the CellTiter-Glo™ Luminescent Assay (Promega Corporation, Madison, Wis.). Cells (5×10³) were added to an opaque-walled 96-well plate. For each reading (day 1, 3, 5, 7 and 9), the plate was equilibrated before adding the CellTiter-Glo® Reagent and luminescence was recorded as described above.

In Vivo Study Using a Tumor Animal Mode

Animal experiments were done according to guidelines governing animal experimentation and approved by the American veterinary authorities. Human MDA-MB-435 invasive cancer cells (2×10⁶) expressing green fluorescent protein (GFP) cDNA were inoculated subcutaneously into mammary tissue of 4-5 week-old female NCR-Nu mice with five mice per group (Taconic). Once palpable, tumors were measured twice/week and volume was calculated using the following formula: length×width×height×0.5236. Mice were treated with a vehicle control (DMSO), compound 4 (20 mg/kg) or compound 2 (20 mg/kg). On alternating days, these compounds were administered by intraperitoneal injection or by direct injection into the tumor site in a volume of 50 μl, 6 days per week. At the end of the experiment, the mice were sacrificed and the tumors and lungs were dissected. Fresh lung sections were cut (˜3 mm thick) and examined for the presence of GFP-expressing tumor foci. Lung metastases were visualized under the microscope using a FITC filter to detect metastatic MDA-MB-435/GFP cells. The area of metastatic foci per field of examination was quantified from 10 random sites of three different slides for each mouse using NIH ImageJ software.

Transwell Chamber Migration Assay, Construction of Plasmids of MMP-9/MMP-2_{PEX}, Gelatin Zymography, Co-Immunoprecipitation, and Three Dimensional (3D) Invasion Assay

These techniques have been described previously (13, 23)

Statistical Analysis

Data is expressed as the mean±standard error of triplicates. Each experiment was repeated as least 3 times. Student's t-test and analysis of variants (ANOVA) were used to assess differences with *P<0.05 (significant), **: P<0.01 (highly significant), and ***: P<0.001 (extremely significant).

### Example 2

### MMP-9 Expression Correlates Survival Probability of Patients

MMP-9 has been identified as one of the Rosetta 70 genes, serving as a poor prognosis signature for patients with breast cancer (24). To gain insight into the clinical significance of MMP-9 in patients with breast cancer, two additional publicly available DNA microarray datasets, which contain a large number of breast cancer patient samples, were analyzed in order to establish a correlation between MMP-9 expression and the probability of disease-free survival.

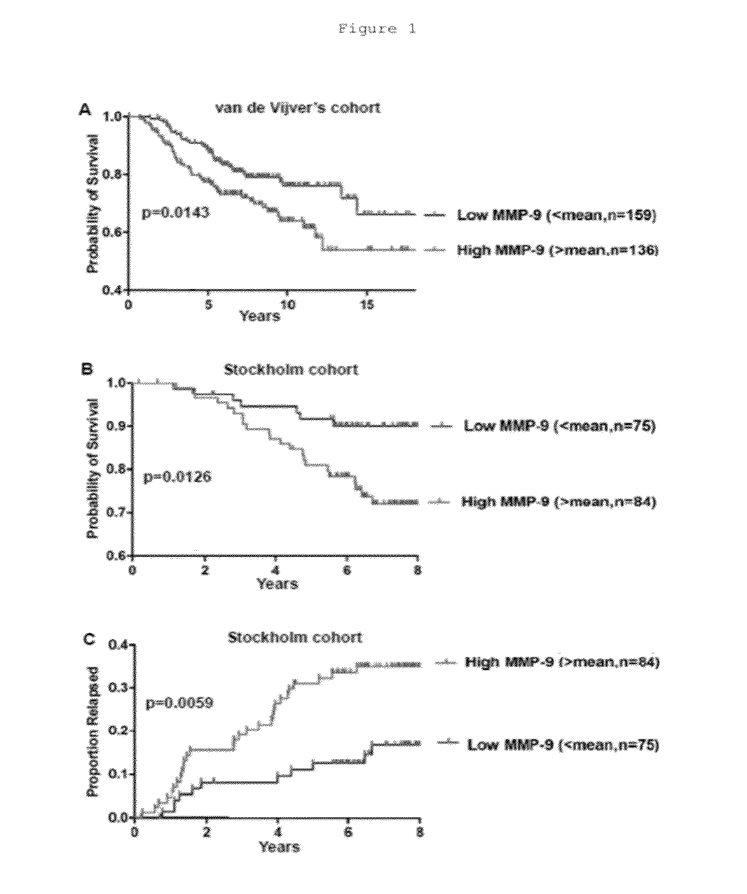

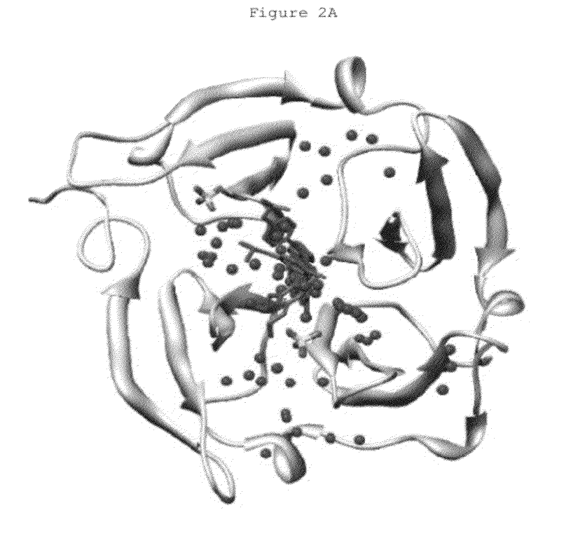

When patient samples were grouped based on MMP-9 RNA expression dichotomized at the mean value in the Van de Vijver cohort, which contains 295 breast cancer patients (25), high expression of MMP-9 was found to be significantly associated with overall survival rate by Kaplan-Meier analysis (P=0.0143) (FIG. 1A). Upon further analysis of lymph node negative group patient samples (120 cases) in the same cohort, based on MMP-9 expression dichotomized at the mean value, high expression of MMP-9 correlated with lower patient survival probability (P=0.0203, data not shown). In analysis of the Stockholm cohort (GSE 1456) (26), similar survival probability results were obtained when MMP-9 RNA expression was dichotomized at the mean from 159 breast cancer patient samples (P=0.0126) (FIG. 1B). In addition, patients in the high MMP-9 expression group had a worse cumulative incidence of relapse (P=0.0059) (FIG. 1C). Hence, elevated expression levels of MMP-9 in breast cancer correlate with a poor prognosis and suppressing MMP-9 may improve patient outcomes.

### Example 3

Identification of Small Molecules Targeting the PEX Domain of MMP-9 Using Dock

A computational docking approach to small molecule discovery utilized DOCK 6.0 (27) to map potential ligand binding sites in the PEX domain of human MMP-9. MMP-9 forms a homodimer, and the dimerization interface is in the PEX domain. The homodimer is observed under X-ray crystallographic conditions (PDB: 1ITV) (19) and in cell culture (12). The structures of the two subunits are similar, but not perfectly symmetrical; therefore subunit A in its entirety was used for docking. A large cavity in the center of the top face of the barrel was identified by DOCK (FIGS. 2A & 8). This cavity, which had been noted previously by Cha and coworkers (19), is formed by the innermost strands of all four blades and the loops which connect them to the second β-strand of each blade.

As proof-of-principle that docking to the PEX domain was feasible, 100 commercially available compounds were selected from the ZINC 2007 database (28) and docked. Molecules which docked to the MMP-9 PEX domain were ranked based on their cluster size, grid score (energies), van der Waals energies and electrostatic energies. Four of five hits (FIG. 2D) contain a 4-pyrimidone core with variable substitutions at the 2- and 6-carbons (FIG. 2C). All five top hits docked to the cavity at the top of the four blades in the PEX domain (FIGS. 2A & B).

### Example 4

### Inhibition of MMP-9-Induced Cell Migration by the Identified Compounds

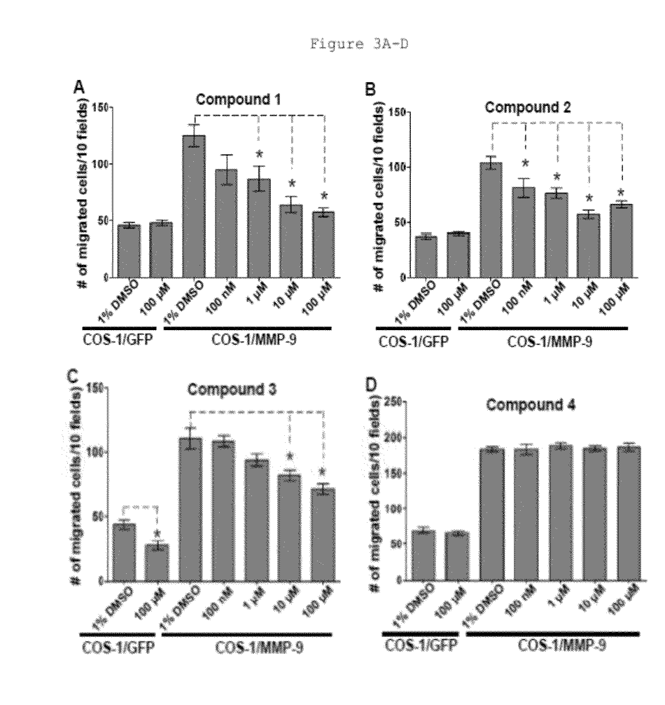

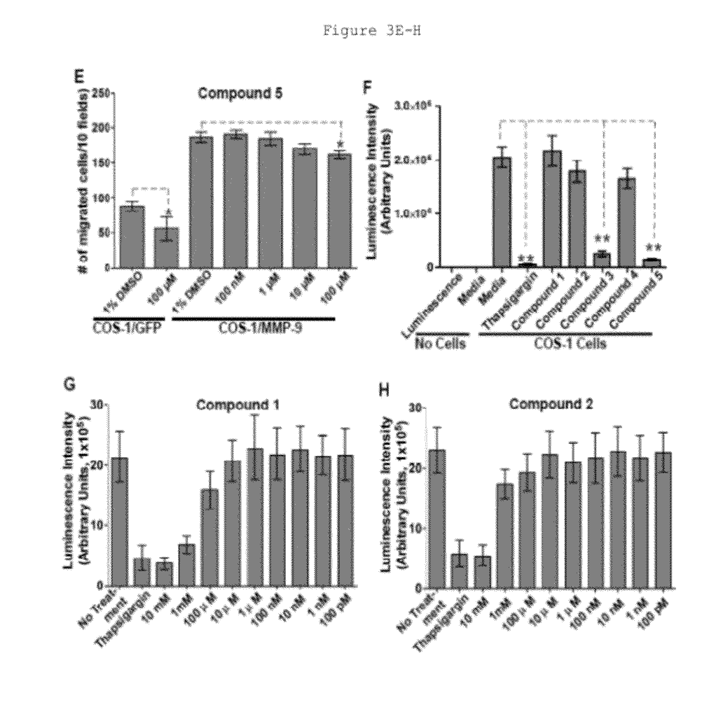

We tested whether the five identified compounds interfered with MMP-9-induced cell migration. COS-1 cells expressing MMP-9 cDNA, or GFP cDNA as a control, were pre-incubated with or without the compounds (doses ranging from 100 nM to 100 μM) for 30 minutes, and examined by a Transwell chamber migration assay. Compounds 1, 2, 3 and 5 inhibited the migration of MMP-9-expressing COS-1 cells, whereas compound 4 showed no activity (FIG. 3A-E). Compounds 3 and 5, but not 1 and 2, inhibited the migration of control cells (GFP-transfected) as well as MMP-9 transfected cells (FIGS. 3C & E).

To rule out the possibility that the reduction of cell migration by these compounds was due to cytotoxicity, a cell viability assay was performed. First, COS-1 cells were treated with a 100 μM dose of each compound for 24 hours followed by a cytotoxicity assay. Thapsigargin, an ER stress inducer that inhibits intracellular Ca²⁺-ATPases (29), was used as a positive control to trigger cell death. Treatment with compounds 1, 2 and 4 did not cause notable cytotoxicity at the maximum concentration used in cell migration assay, whereas treatment with compounds 3 and 5 induced cell death (FIG. 3F).

Second, the lethal dose (LD₅₀) of compounds 1 and 2 was determined in COS-1 cells. Cells were treated with increasing doses of the compounds for 24 hours followed by cell viability assay (FIGS. 3G & H). The LD₅₀ of compounds 1 and 2 were 360±2 μM and 3.5±0.3 mM, respectively, suggesting that their inhibition of MMP-9-induced cell migration was not due to their cytotoxicity.

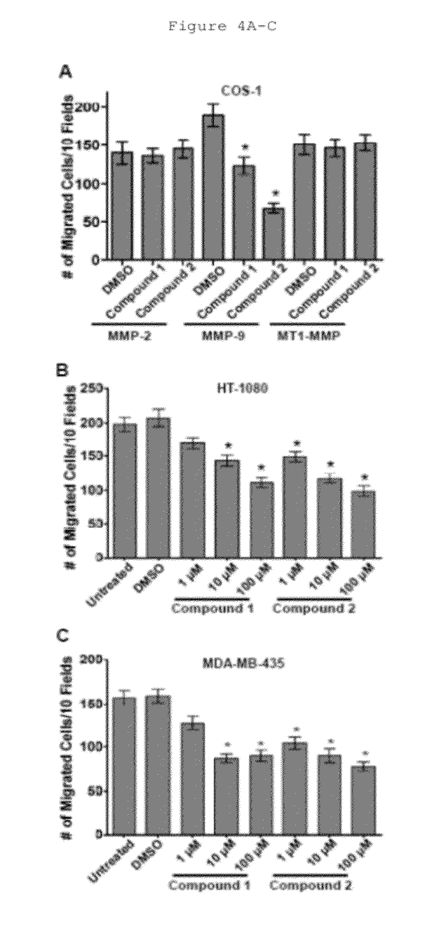

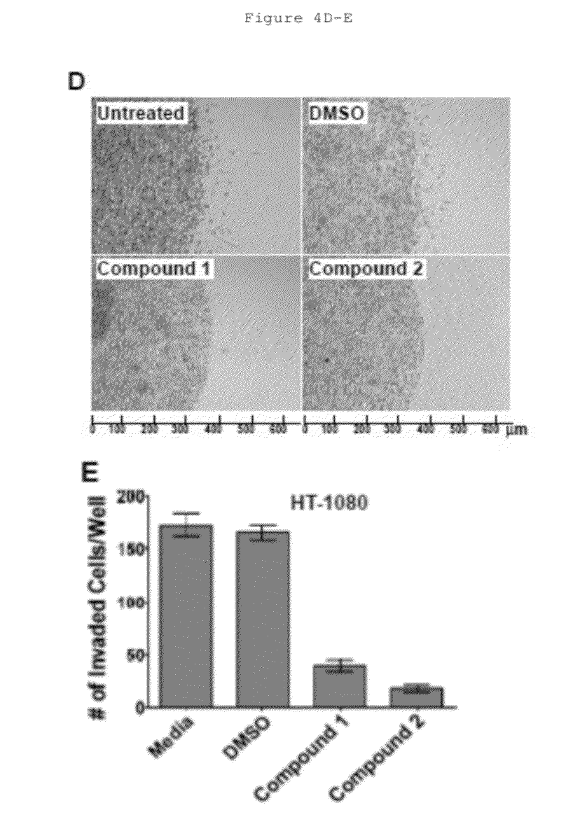

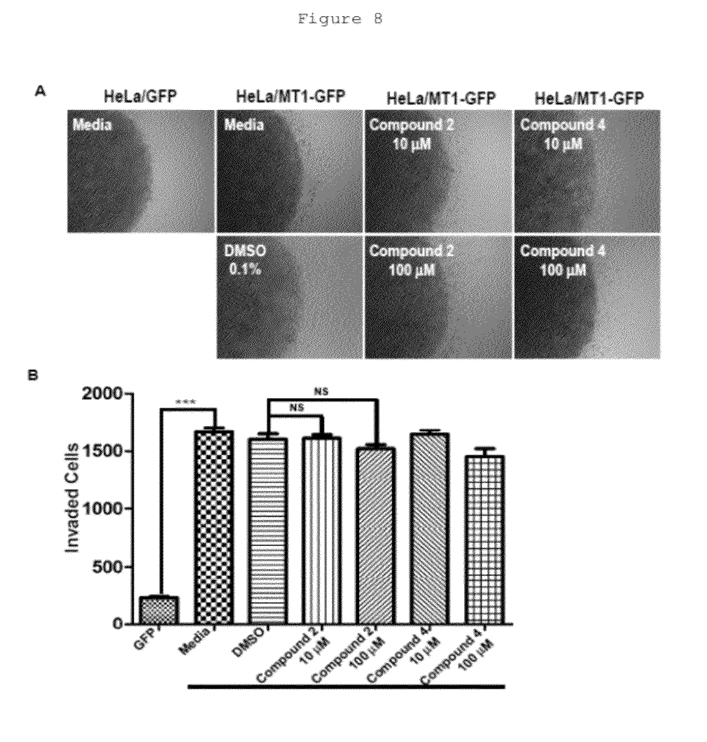

To further determine the specificity and selectivity of compounds 1 and 2 for MMP-9-induced cell migration, their effect on cell migration induced by other MMPs, e.g., MMP-2 and MT1-MMP (membrane type 1-MMP; MMP-14) was examined, in which the PEX domain has been reported to play a critical role in enhanced cell migration (30). COS-1 cells ectopically expressing MMP-2 or MT1-MMP cDNA were examined for their cell migratory abilities (Transwell chamber migration assay) in the presence or absence of compound 1 or 2. In contrast to MMP-9 expressing cells, neither compound inhibited the migration of MMP-2 or MT1-MMP expressing COS-1 cells (FIG. 4A). In addition, compound 2 did not interfere with MT1-MMP-mediated cancer cell invasion examined by a 3D invasion assay (FIG. 8). Thus, small synthetic compounds that potentially bind to the PEX domain of MMP-9 inhibit MMP-9-induced cell migration with enhanced-specificity and -selectivity.

### Example 5

### Inhibition of Migration in Cancer Cells that Produce Endogenous MMP-9 by Compounds 1 and 2

Compounds 1 and 2 were investigated for inhibition of migration in cells producing a pathologically relevant level of endogenous MMP-9. Two human invasive cancer cell lines, HT-1080 and MDA-MB-435, expressing high endogenous levels of MMP-9 were employed. HT-1080 and MDA-MB-435 cells were incubated with compounds 1 and 2 at concentrations ranging from 1 μM to 100 μM for 30 minutes before assay for Transwell chamber migration. Both compounds inhibited the migration of HT-1080 and MDA-MB-435 cells in a dose-dependent manner (FIGS. 4B & C).

Because cell migration is a critical determinant of cancer cell invasiveness, compounds 1 and 2 were examined for interference of cancer cell invasion through inhibition of cell migration. To this end, HT-1080 cells were assessed in the 3D type I collagen invasion assay (23). As anticipated, the cell invasive ability of HT-1080 cells was significantly inhibited in cells treated with compounds 1 and 2 (FIGS. 4D & E). Inhibition of MDA-MB-435 cell invasion was also observed (data not shown). These data suggest that inhibition of MMP-9-mediated cell migration by compound 2 results in suppressed cancer cell invasion.

### Example 6

### Compounds 1 and 2 do not Affect MMP-9 Expression or Proteolytic Activity

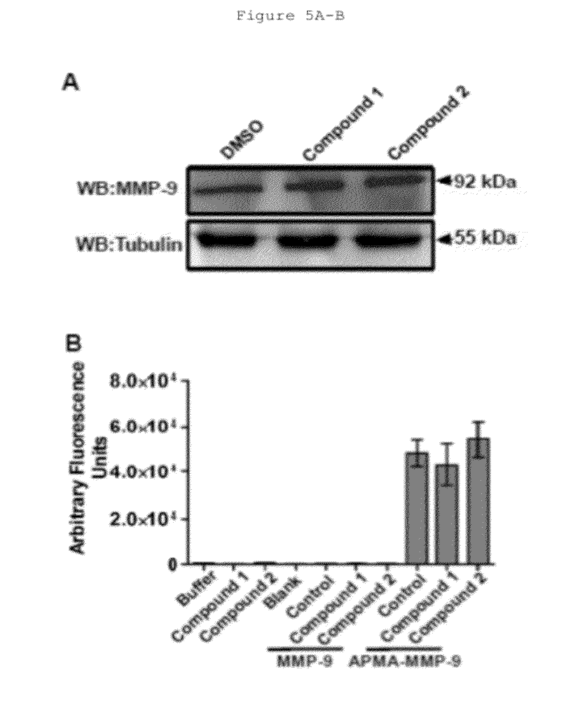

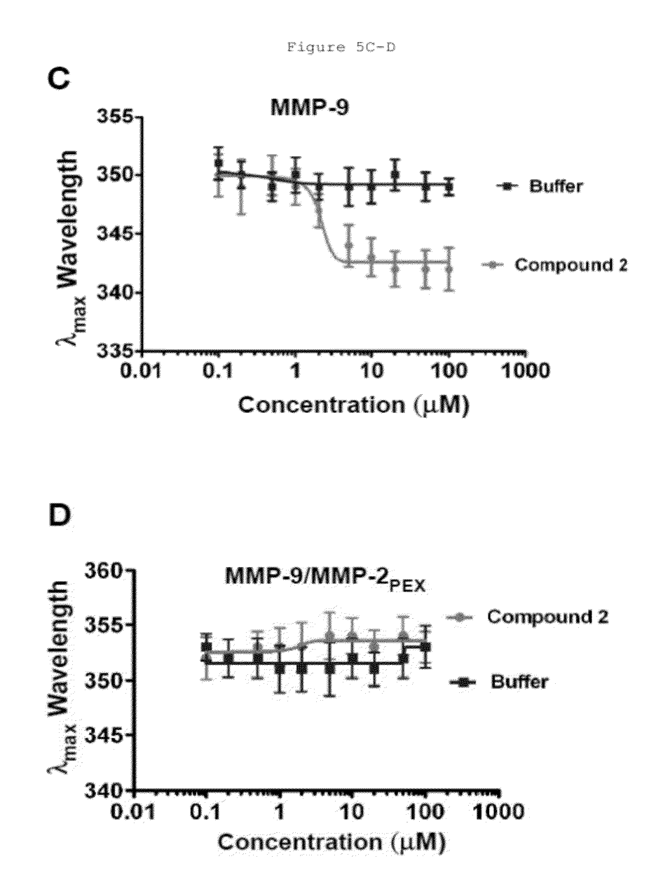

Interference with MMP-9 expression by compounds 1 and 2 was examined by a Western blot using an anti-MMP-9 antibody. Secreted MMP-9 in the conditioned medium from HT-1080 cells in the presence or absence of compounds 1 and 2 was tested. Western blotting using an antibody to tubulin was also employed as a control. No effect on MMP-9 expression by the compounds was observed through Western blotting (FIG. 5A).

A fluorogenic peptide assay using Mca-P-L-G-L-Dpa-A-R-NH₂ as a substrate was utilized to determine if compounds 1 and 2 could disrupt MMP-9 catalytic activity (21). The conditioned media of COS-1 cells transfected with MMP-9 cDNA was collected. As described (12), activated MMP-9 was obtained by incubating proMMP-9 derived from the conditioned media of COS-1 cells transfected with MMP-9 cDNA with p-aminophenyl mercuric acetate (APMA). Addition of compounds 1 and 2 to APMA-activated MMP-9 did not inhibit the catalytic activity of MMP-9 as measured by cleavage of the fluorescent MCA peptide (FIG. 5B). These data suggest that inhibition of MMP-9-induced cell migration by compounds 1 and 2 is not due to inhibition of MMP-9 expression or proteolytic activity.

### Example 7

### Binding of Compound 2 to the MMP-9 PEX Domain

The physical interaction of compound 2 with MMP-9 was characterized. The change in MMP-9 fluorescence was titrated, which contains 14 tryptophans per monomer, upon addition of compound 2. The emission from 290 to 450 nm with excitation at 280 nm was recorded. Saturation of purified proMMP-9 with compound 2 resulted in a 7 nm blue shift in the λₘₐₓ of MMP-9 emission (FIG. 5C). No effect on the protein fluorescence occurred in the buffer-only control. The K_{d} for MMP-9 binding to compound 2 is 2.1±0.2 μM.

To further characterize the binding between compound 2 and MMP-9, a previously generated chimera of MMP-9 was employed in which the PEX domain of MMP-9 was replaced with that of MMP-2 (MMP-9/MMP-2_{PEX}) (13). Upon addition of compound 2 to MMP-9/MMP-2_{PEX}, no shift in fluorescence was detected (FIG. 5D). Similar result was also obtained between compound 2 with purified recombinant soluble MT1-MMP (31) (data not shown).

These data confirmed that compound 2 binds specifically to the PEX domain of MMP-9. The absorption of compound 1 at 280 nm precluded evaluation of its binding properties.

MMP-9 homodimerization as a prerequisite for enhanced cell migration was previously demonstrated (13). This observation led to hypothesis that compound 2 bound to the PEX domain, might act as an inhibitor of proMMP-9 homodimerization, thus, interfering with MMP-9-induced cell migration. To test this hypothesis, COS-1 cells transfected with both proMMP-9/Myc and proMMP-9/HA cDNAs in the presence or absence of compounds 2 and 4 were examined by co-immunoprecipitation assay as described previously (13). As shown in FIG. 5E, treatment of the transfected cells with compound 2, but not inactive compound 4, resulted in blocked MMP-9 homodimer formation. This defect was not due to inhibition of expression of MMP-9 by compound 2 as evidenced by Western blotting of the cell lysate from HT-1080 cells (FIG. 5A). Similar results were obtained in reciprocal coimmunoprecipitation assays in which Myc-tagged MMP-9 was immunoprecipitated and HA-tagged MMP-9 was examined by Western blotting (FIG. 5E). This reverse approach confirms that compound 2 specifically affects MMP-9 homodimerization.

It has been reported that homodimerized MMP-9 interacts with cell surface adhesion molecule, CD44, which leads to activation of EGFR and downstream MAPK (ERK1/2) pathway (12, 13). The activity status of downstream effector ERK1/2 was examined to determine if compound 2 interrupts this signaling pathway in MMP-9-mediated cell migration. COS-1 cells ectopically expressing MMP-9 cDNA were serum starved in the presence or absence of compounds for 18 hours followed by Western blotting using anti-phosphor-ERK1/2 and total ERK1/2 antibodies. As depicted in FIG. 5F, decreased activation of ERK1/2 was observed in compound 2 treated cells. Taken together, these data suggest that abrogation of MMP-9-mediated cell migration by compound 2 is due to disruption of MMP-9 homodimerization, which results in failure to cross-talk with CD44 and the EGFR-MAPK signaling pathway.

### Example 8

### Effect on MMP-9-Mediated Cell Proliferation by Compound 2

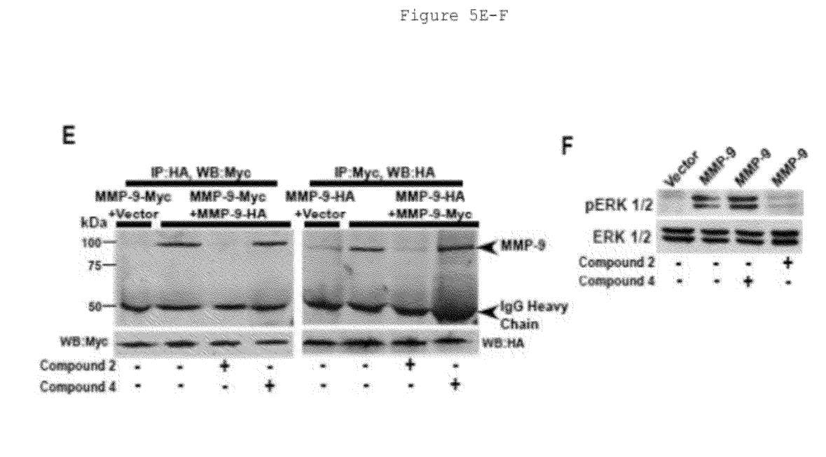

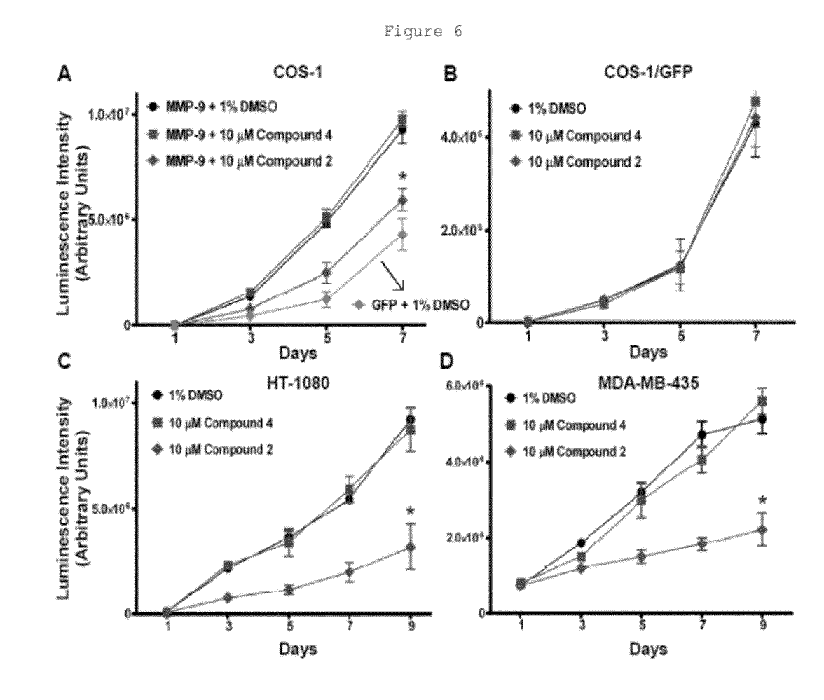

MMP-9 has been linked to increased cell proliferation (32). To investigate whether the selected small molecules affect MMP-9-mediated cell proliferation, COS-1 cells transfected with MMP-9 cDNA or GFP cDNA (control) were monitored for proliferation in the absence or presence of compound 2 (10 μM) or compound 4 (10 μM. no effect on MMP-9-induced cell migration) with a CellTiter-Glo® Luminescent assay. In agreement with previous observations (32), the rate of cell proliferation increased significantly (P<0.05) in COS-1 cells expressing MMP-9 as compared to GFP expressing COS-1 cells (FIG. 6A). MMP-9-induced cell proliferation was not affected by compound 4, consistent with its lack of effect on MMP-9-induced cell migration. In contrast, compound 2 significantly decreased MMP-9-induced cell proliferation (FIG. 6A), but did not affect the proliferation of COS-1 cells transfected with GFP cDNA (FIG. 6B). To determine if compound 2 also affects the proliferation of cancer cells producing endogenous MMP-9, HT-1080 and MDA-MB-435 cancer cells were treated with 10 μM solution of compound 2. Significant inhibition of cell proliferation was observed for HT-1080 and MDA-MB-435 cells treated with compound 2, but not with compound 4 or with DMSO controls (FIGS. 6C & D).

### Example 9

### Decreased Tumor Growth and Lung Metastases in Compound 2-Treated Mice

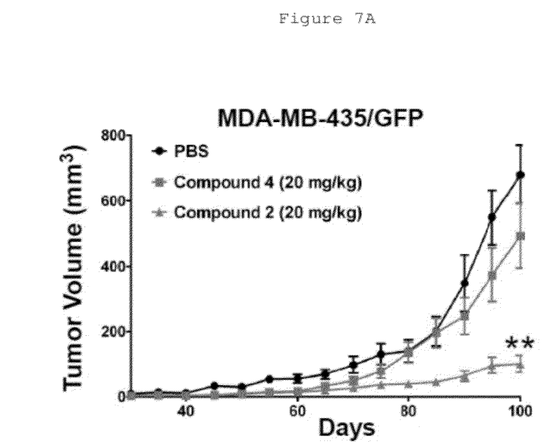

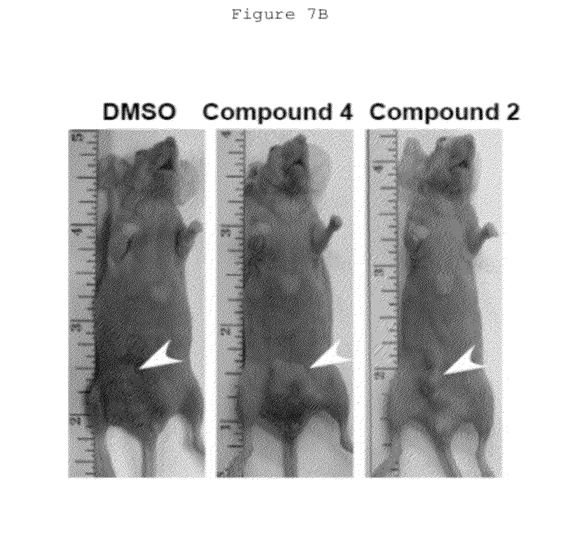

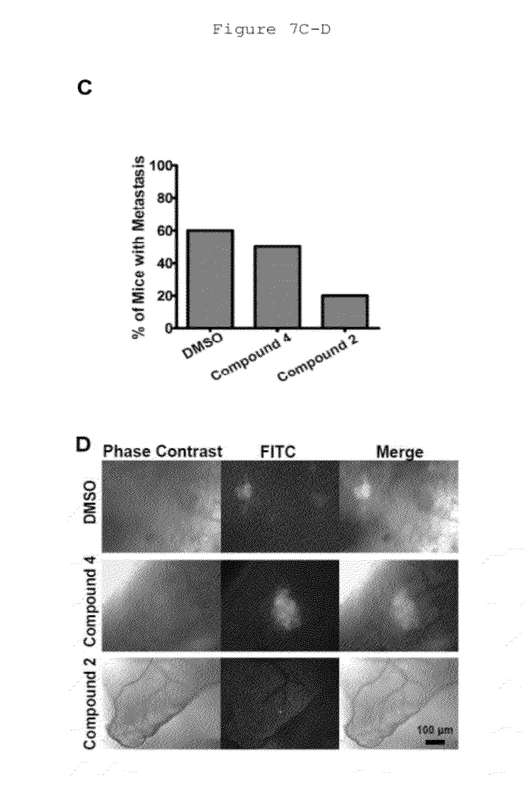

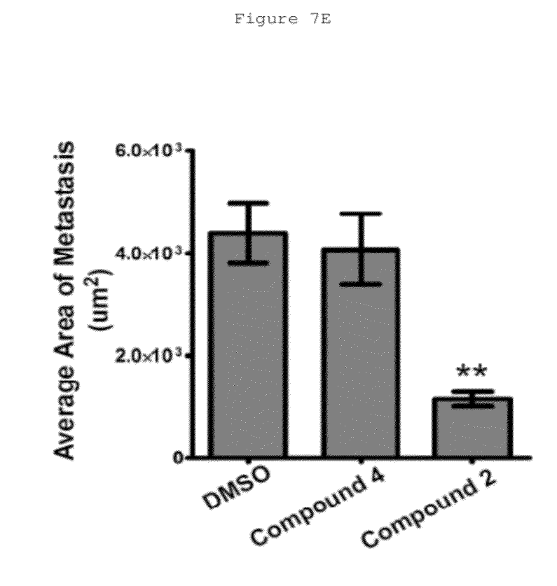

Based on its efficacy at inhibiting the in vitro migration and proliferation of MMP-9 transfected COS-1 cells, the in vivo effects of compound 2 on MDA-MB-435 cancer cells was explored. MDA-MB-435 cancer cells produce a high level of endogenous MMP-9 and possess a high metastatic potential (33-35). To facilitate in vivo analysis of tissues and visualization of the lung metastases, MDA-MB-435 cells were stably transfected with GFP cDNA and implanted subcutaneously within the mammary fat pad of female immunodeficient mice. Treatment of mice with compound 2 resulted in a profound delay in tumor growth, whereas treatment with the inactive control compound 4 or the vehicle alone failed to inhibit tumor growth (FIGS. 7A & B). Tumor incidence was unaffected by compound 2.

To analyze the effects of compound 2 on metastasis, the lungs of tumor-bearing mice were removed and slices of the lungs (3 mm thickness) were examined under a fluorescent microscope (FIG. 7C). In the vehicle control and compound 4-treated groups, multiple large nodules were evident in MDA-MB-435/GFP tumor-bearing mice, whereas the extent of lung metastasis was dramatically reduced in mice treated with compound 2 (FIG. 7C). Also, dimensions of tumor foci area in the lung and the percent of mice displaying lung metastases were significantly decreased in these mice (FIGS. 7D & E). Thus, treatment with compound 2 impaired the in vivo effect of MMP-9 on both primary tumor growth and metastasis. No significant change in body weight nor other signs of toxicity during the 14-week period were observed in compound 2-treated mice.

Discussion

Although continuous progress has been made in the identification of molecules involved in metastasis, the contributions and timing of key regulatory molecules remain unclear (36). This has delayed development of effective treatment strategies targeting metastasis. Regardless of which molecules initiate metastasis, considerable evidence indicates that cancer cells require proteases, including MMPs, for their invasive behavior (3). Targeting MMPs has been implicated as a viable approach to inhibit cancer dissemination. Given the fact that the catalytic-core binding sites among all MMPs are highly conserved, targeting non-catalytic sites of MMPs may increase target selectivity and/or specificity. In this study, it was demonstrated that certain small molecule synthetic compounds specifically interfere with MMP-9-mediated cell migration. This inhibitory effect works through the abrogation of MMP-9 dimerization via the PEX domain, and subsequent blockage of the CD44-EGFR-MAKP signaling pathway (13).

The role of the PEX domain of MMP-9 in cancer cell migration has gained considerable attention (37-39). Lengyel et al. (40) demonstrated that high expression of proMMP-9 in ovarian cancer patients correlated with poor survival; activated MMP-9 was not implicated or detected, thus suggesting a critical role of the non-catalytic domains of MMP-9 in cancer progression. It was previously demonstrated that latent MMP-9 was able to initiate cell migration independent of its catalytic activity and that it works through a proMMP-9-CD44-EGFR-MAPK pathway (12, 13). It was identified that the PEX domain plays a critical role in proMMP-9-mediated cell migration, which is in agreement with previous reports (37-39). The validation of the MMP-9 PEX domain as a drug target has recently been explored through different experimental approaches: 1) anti-PEX antibodies block Schwann cell migration (38); 2) exogenous recombinant PEX domain inhibits the migration of endothelial cells (anti-angiogenic effect), as well as intracranial glioblastoma growth (37); 3) non-catalytic-domain inhibitors of MMP-9, selected from a phage display peptide library, interfere with cell migration and tumor formation (39); and 4) peptides mimicking motifs of the outermost strands of the first and fourth blades of the PEX domains of MMP-9 abrogate MMP-9-mediated cell migration (13). These results prompted us to hypothesize that low molecular weight compounds interacting with the PEX domain of MMPs may interfere with its function.

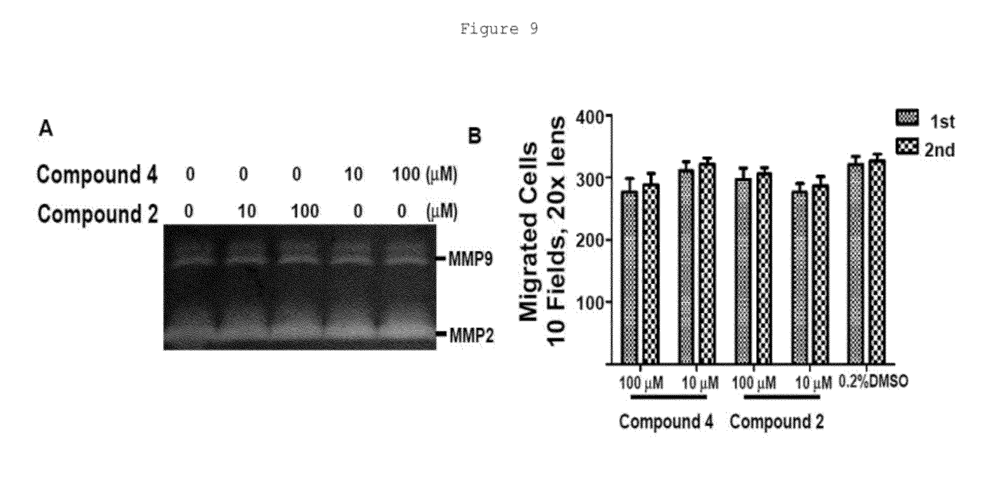

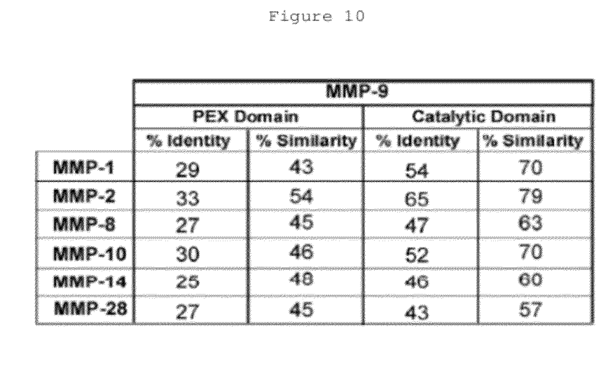

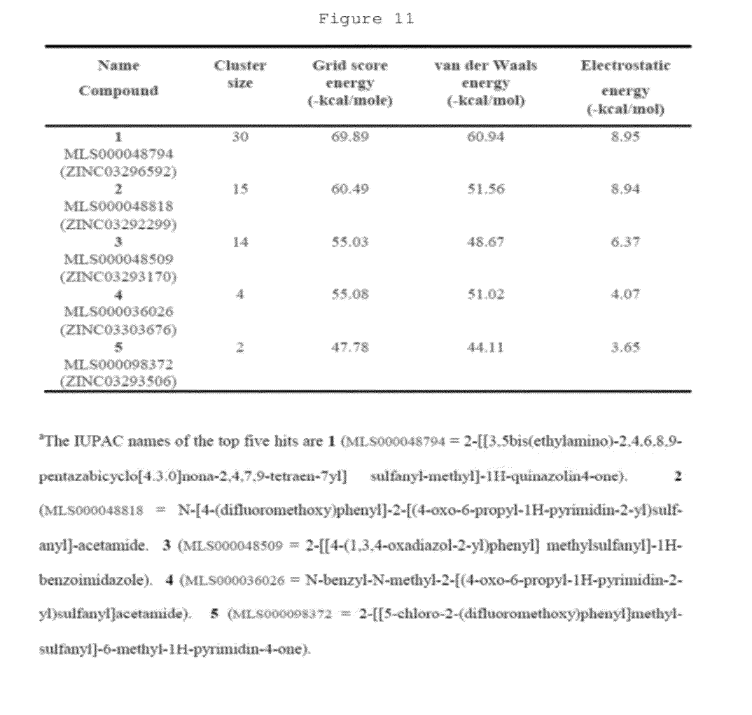

The PEX domain of MMP-9 shares low amino acid identity (25% to 33%) as contrast to the catalytic domain (43-65%), with secreted MMP-1, -2, -8, -9, -10, and -28 as well as membrane anchored MT1-MMP (FIG. 10) as determined by BLAST (bl2seq) alignment analysis (National Center for Biotechnology Information). The genetic differences between the PEX domains of MMP-9 and other MMPs determine their distinct biological roles in cell function.

The MMP-9 PEX domain is a barrel-shaped structure composed mostly of hydrophobic surfaces making this domain difficult to target with drugs (5, 19). Our in silico results indicate that the surface cavity within the loops of the MMP-9 PEX domain appears to be targetable by small compounds (FIG. 2B). The structural components of the best three compounds each contain a six-membered heterocycle which docks within the identified site in the PEX domain. The flat rings preferentially bind within the cavity of the MMP-9 PEX domain blades, whereas the more flexible side chains (i.e. arylamide group of compound 2) likely bind on the surface proximal to the cavity (FIG. 2B). MMP-9 homodimerizes through interactions in the fourth blade of its PEX domain (19). Furthermore, formation of the homodimer is required for cell migration, an important component of metastasis. Blade III is flexible due to the presence of Gly⁶¹⁵ and its flexibility may contribute to stabilization of the monomeric versus the dimeric form of the PEX domain (19). Therefore, it was reasoned that compounds bound in the cavity of the PEX domain may allosterically disrupt the dimer interface and consequently migration.

Indeed, four of the five best docking compounds, identified through in silica analysis, inhibited MMP-9-induced cell migration. Compounds 1 and 2 specifically and selectively inhibited cell migration induced either by ectopically expressed MMP-9 or by endogenous MMP-9 as well as cancer cell invasion in a 3D type I collagen invasion assay (FIG. 4D). The latter is believed to be due to the inhibition of cell migration, because cell migration is a prerequisite for cancer invasion. These inhibitory activities are independent of an effect on MMP-9 catalytic activity, which suggested that compound 2 physically interacts with the PEX domain of MMP-9. The interaction of compound 2 with the PEX domain was further validated using a biophysical method. Based on quenching of MMP-9 tryptophan fluorescence, it was determined that compound 2 specifically binds to the PEX domain of MMP-9, but not to the PEX domain of MMP-2 (FIGS. 5C & D) (The interaction of compound 1 with PEX domain of MMP-9 was not confirmed because of its interfering spectroscopic properties). Importantly, consistent with our in vitro studies, compound 2 inhibited cancer cell metastasis in vivo (FIG. 7), presumably due to reduction in cell proliferation, angiogenesis and migration. To our knowledge, compound 2 has not been previously reported to possess activity against human cancers or MMPs.

MMP-9 has been implicated in primary tumor growth and metastasis of various cancers (2). Silencing MMP-9 in cancer cells was reported to reduce tumor growth, angiogenesis, invasion and metastasis (41). However, there are inconsistencies in the literature regarding whether or not MMP-9 enhances tumor growth in in vivo cancer models (1, 42, 43). In our study, compound 2 inhibited tumor growth without affecting tumorigenicity. It was also observed that compound 2 inhibited tumor metastasis of MDA-MB-435/GFP cells in vivo, presumably by interfering with PEX interaction(s) with downstream pathway(s) required for cell migration and invasion. In agreement with previous studies (37, 39), our results suggest that the PEX domain of MMP-9 is involved in both cell proliferation and migration which contribute to increased tumor growth and metastasis. Importantly, it has been demonstrated that the main source of MMP-9 in some murine tumor models is from tumor-associated stromal and inflammatory cells (1, 32, 42, 44), leading to the possibility that anti-tumor effect of an MMP-9 inhibitor in vivo could be due to an effect on host cells. Human MMP-9 and murine MMP-9 proteins share 72% amino acid identity. To distinguish whether the anti-tumor activity of compound 2 in vivo is due to an effect on mouse stromal/inflammatory cells or on human tumor cells, the effect of this compound on mouse RAW264.7 macrophage-like cells expressing endogenous MMP-9 was examined. Compound 2 had no notable effect on the migration of mouse macrophages (FIG. 9), leading us to conclude that the inhibitory effect of compound 2 is directed at human tumor MMP-9.

Regarding the provenance of the MDA-MB-435 cell line, these cells were initially derived from the pleural effusion of a female patient with breast cancer (34, 45), and have been extensively used as an experimental model of breast cancer. A recent DNA microarray study, however, indicated that this cell line clustered with several melanoma cell lines (46), leading to speculation that MDA-MB-435 cells might be of melanoma, rather than breast in origin (47). Another report demonstrated that MDA-MB-435 is most likely a breast epithelial cell line that has undergone lineage infidelity (48). Regardless of its origin, MDA-MB-435 cells are highly metastatic in nude mice (34, 45) and produce high levels of MMP-9, thus serving as an appropriate experimental model to explore the inhibitory activity of compounds exhibiting anti-MMP-9 activity.

It was previously demonstrated that CD44 is a key molecule involved in proMMP-9 enhanced cell migration through an EGFR-CD44 signaling pathway along with other downstream effectors including phosphorylated FAK, AKT and ERK (13). Since compound 2 inhibits MMP-9 dimerization and ERK1/2 activation, it was proposed that the mechanism of inhibition by compound 2 of MMP-9-mediated cell migration is via interfering with MMP-9 dimer formation, and hence, blockage of the downstream MMP-9-CD44-EGFR-MAPK signaling pathway. In accordance with our data, Peng et al. (49) demonstrated that an increase in MMP-9, after colocalization with CD44 on the cell surface of MDA-MB-435 cells, stimulated the tumor cell invasion and metastasis. In another study, Yu and Stamenkovic (50) demonstrated a cascade involving a MMP-9/CD44/TGF-β pathway that promotes the metastatic potential of selected tumor types; interference with the MMP-9/CD44 complex induced apoptosis.

### REFERENCES

1. Acuff H B, Carter K J, Fingleton B, Gorden D L, Matrisian L M. Matrix metalloproteinase-9 from bone marrow-derived cells contributes to survival but not growth of tumor cells in the lung microenvironment. Cancer Res. 2006; 66:259-66.
2. Deryugina E I, Quigley J P. Matrix metalloproteinases and tumor metastasis. Cancer Metastasis Rev. 2006; 25:9-34.
3. Kessenbrock K, Plaks V, Werb Z. Matrix metalloproteinases: regulators of the tumor microenvironment. Cell. 2010; 141:52-67.
4. Overall C M, Kleifeld O. Tumour microenvironment—opinion: validating matrix metalloproteinases as drug targets and anti-targets for cancer therapy. Nat Rev Cancer. 2006; 6:227-39.
5. Overall C M, Kleifeld O. Towards third generation matrix metalloproteinase inhibitors for cancer therapy. Br J Cancer. 2006; 94:941-6.
6. Hidalgo M, Eckhardt S G. Development of matrix metalloproteinase inhibitors in cancer therapy. J Natl Cancer Inst. 2001; 93:178-93.
7. Whittaker M, Floyd C D, Brown P, Gearing A J. Design and therapeutic application of matrix metalloproteinase inhibitors. Chem. Rev. 1999; 99:2735-76.
8. Zucker S, Cao J, Chen W T. Critical appraisal of the use of matrix metalloproteinase inhibitors in cancer treatment. Oncogene. 2000; 19:6642-50.
9. Overall C M, Lopez-Otin C. Strategies for MMP inhibition in cancer: innovations for the post-trial era. Nat Rev Cancer. 2002; 2:657-72.
10. McQuibban G A, Gong J H, Tam E M, McCulloch C A, Clark-Lewis I, Overall C M. Inflammation dampened by gelatinase A cleavage of monocyte chemoattractant protein-3. Science. 2000; 289:1202-6.
11. Tam E M, Wu Y I, Butler G S, Stack M S, Overall C M. Collagen binding properties of the membrane type-1 matrix metalloproteinase (MT1-MMP) hemopexin C domain. The ectodomain of the 44-kDa autocatalytic product of MT1-MMP inhibits cell invasion by disrupting native type I collagen cleavage. J Biol. Chem. 2002; 277:39005-14.
12. Dufour A, Sampson N S, Zucker S, Cao J. Role of the hemopexin domain of matrix metalloproteinases in cell migration. J Cell Physiol. 2008; 217:643-51.
13. Dufour A, Zucker S, Sampson N S, Kuscu C, Cao J. Role of matrix metalloproteinase-9 (MMP-9) dimers in cell migration: design of inhibitory peptides. J Biol. Chem. 2010.
14. Lee M, Fridman R, Mobashery S. Extracellular proteases as targets for treatment of cancer metastases. Chem Soc Rev. 2004; 33:401-9.
15. Bjorklund M, Koivunen E. Gelatinase-mediated migration and invasion of cancer cells. Biochim Biophys Acta. 2005; 1755:37-69.
16. Coussens L M, Fingleton B, Matrisian L M. Matrix metalloproteinase inhibitors and cancer: trials and tribulations. Science. 2002; 295:2387-92.
17. Sanceau J, Truchet S, Bauvois B. Matrix metalloproteinase-9 silencing by RNA interference triggers the migratory-adhesive switch in Ewing's sarcoma cells. J Biol. Chem. 2003; 278:36537-46.
18. Berman H, Henrick K, Nakamura H. Announcing the worldwide Protein Data Bank. Nat. Struct Biol. 2003; 10:980.
19. Cha H, Kopetzki E, Huber R, Lanzendorfer M, Brandstetter H. Structural basis of the adaptive molecular recognition by MMP9. J Mol. Biol. 2002; 320:1065-79.
20. Pettersen E F, Goddard T D, Huang C C, Couch G S, Greenblatt D M, Meng E C, et al. UCSF Chimera—a visualization system for exploratory research and analysis. J Comput Chem. 2004; 25:1605-12.
21. Knight C G, Willenbrock F, Murphy G. A novel coumarin-labelled peptide for sensitive continuous assays of the matrix metalloproteinases. FEBS Lett. 1992; 296:263-6.
22. Toth M, Gervasi D C, Fridman R. Phorbol ester-induced cell surface association of matrix metalloproteinase-9 in human MCF10A breast epithelial cells. Cancer Res. 1997; 57:3159-67.
23. Cao J, Chiarelli C, Richman O, Zarrabi K, Kozarekar P, Zucker S. Membrane type 1 matrix metalloproteinase induces epithelial-to-mesenchymal transition in prostate cancer. J Biol Chem. 2008; 283:6232-40.
24. van 't Veer L J, Dai H, van de Vijver M J, He Y D, Hart A A, Mao M, et al. Gene expression profiling predicts clinical outcome of breast cancer. Nature. 2002; 415:530-6.
25. van de Vijver M J, He Y D, van't Veer L J, Dai H, Hart A A, Voskuil D W, et al. A gene-expression signature as a predictor of survival in breast cancer. N Engl J. Med. 2002; 347:1999-2009.
26. Pawitan Y, Bjohle J, Amler L, Borg A L, Egyhazi S, Hall P, et al. Gene expression profiling spares early breast cancer patients from adjuvant therapy: derived and validated in two population-based cohorts. Breast Cancer Res. 2005; 7:R953-64.
27. Harkcom W T, Bevan D R. Molecular docking of inhibitors into monoamine oxidase B. Biochem Biophys Res Commun. 2007; 360:401-6.
28. Irwin J J, Shoichet B K. ZINC—a free database of commercially available compounds for virtual screening. J Chem Inf Model. 2005; 45:177-82.
29. Baffy G, Miyashita T, Williamson J R, Reed J C. Apoptosis induced by withdrawal of interleukin-3 (IL-3) from an IL-3-dependent hematopoietic cell line is associated with repartitioning of intracellular calcium and is blocked by enforced Bcl-2 oncoprotein production. J Biol. Chem. 1993; 268:6511-9.
30. Cao J, Kozarekar P, Pavlaki M, Chiarelli C, Bahou W F, Zucker S. Distinct roles for the catalytic and hemopexin domains of membrane type 1-matrix metalloproteinase in substrate degradation and cell migration. J Biol Chem. 2004; 279:14129-39.
31. Cao J, Sato H, Takino T, Seiki M. The C-terminal region of membrane type matrix metalloproteinase is a functional transmembrane domain required for pro-gelatinase A activation. J Biol. Chem. 1995; 270:801-5.
32. Coussens L M, Tinkle C L, Hanahan D, Herb Z. MMP-9 supplied by bone marrow-derived cells contributes to skin carcinogenesis. Cell. 2000; 103:481-90.
33. Khaldoyanidi S K, Glinsky V V, Sikora L, Glinskii A B, Mossine V V, Quinn T P, et al. MDA-MB-435 human breast carcinoma cell homo- and heterotypic adhesion under flow conditions is mediated in part by Thomsen-Friedenreich antigen-galectin-3 interactions. J Biol. Chem. 2003; 278:4127-34.
34. Price J E. Metastasis from human breast cancer cell lines. Breast Cancer Res Treat. 1996; 39:93-102.
35. Seraj M J, Samant R S, Verderame M F, Welch D R. Functional evidence for a novel human breast carcinoma metastasis suppressor, BRMS1, encoded at chromosome 11q13. Cancer Res. 2000; 60:2764-9.
36. Coghlin C, Murray G I. Current and emerging concepts in tumour metastasis. J Pathol. 2010; 222:1-15.
37. Ezhilarasan R, Jadhav U, Mohanam I, Rao J S, Gujrati M, Mohanam S. The hemopexin domain of MMP-9 inhibits angiogenesis and retards the growth of intracranial glioblastoma xenograft in nude mice. Int J Cancer. 2009; 124:306-15.
38. Mantuano E, Inoue G, Li X, Takahashi K, Gaultier A, Gonias S L, et al. The hemopexin domain of matrix metalloproteinase-9 activates cell signaling and promotes migration of schwann cells by binding to low-density lipoprotein receptor-related protein. J. Neurosci. 2008; 28:11571-82.
39. Bjorklund M, Heikkila P, Koivunen E. Peptide inhibition of catalytic and noncatalytic activities of matrix metalloproteinase-9 blocks tumor cell migration and invasion. J Biol. Chem. 2004; 279:29589-97.
40. Lengyel E, Schmalfeldt B, Konik E, Spathe K, Harting K, Fenn A, et al. Expression of latent matrix metalloproteinase 9 (MMP-9) predicts survival in advanced ovarian cancer. Gynecol Oncol. 2001; 82:291-8.
41. Sun Y, Liu M, Yang B, Lu J, Li B. Inhibition of laryngeal cancer cell invasion and growth with lentiviral-vector delivered short hairpin RNA targeting human MMP-9 gene. Cancer Invest. 2008; 26:984-9.
42. Bergers G, Brekken R, McMahon G, Vu T H, Itoh T, Tamaki K, et al. Matrix metalloproteinase-9 triggers the angiogenic switch during carcinogenesis. Nat Cell Biol. 2000; 2:737-44.
43. Chattopadhyay S, Shubayev V I. MMP-9 controls Schwann cell proliferation and phenotypic remodeling via IGF-1 and ErbB receptor-mediated activation of MEK/ERK pathway. Glia. 2009; 57:1316-25.
44. Hiratsuka S, Nakamura K, Iwai S, Murakami M, Itoh T, Kijima H, et al. MMP9 induction by vascular endothelial growth factor receptor-1 is involved in lung-specific metastasis. Cancer Cell. 2002; 2:289-300.
45. Price J E, Zhang R D. Studies of human breast cancer metastasis using nude mice. Cancer Metastasis Rev. 1990; 8:285-97.
46. Ross D T, Scherf U, Eisen M B, Perou C M, Rees C, Spellman P, et al. Systematic variation in gene expression patterns in human cancer cell lines. Nat. Genet. 2000; 24:227-35.
47. Chambers A F. MDA-MB-435 and M14 cell lines: identical but not M14 melanoma? Cancer Res. 2009; 69:5292-3.
48. Sellappan S, Grijalva R, Zhou X, Yang W, Eli M B, Mills G B, et al. Lineage infidelity of MDA-MB-435 cells: expression of melanocyte proteins in a breast cancer cell line. Cancer Res. 2004; 64:3479-85.
49. Peng S T, Su C H, Kuo C C, Shaw C F, Wang H S. CD44 crosslinking-mediated matrix metalloproteinase-9 relocation in breast tumor cells leads to enhanced metastasis. Int J Oncol. 2007; 31:1119-26.
50. Yu Q, Stamenkovic I. Transforming growth factor-beta facilitates breast carcinoma metastasis by promoting tumor cell survival. Clin Exp Metastasis. 2004; 21:235-42.

What is claimed is:

1. A method of inhibiting matrix metalloproteinase 9 (MMP-9) dimerization without substantially inhibiting the catalytic activity of MMP-9, comprising contacting the MMP-9 with a small molecule compound of the structure 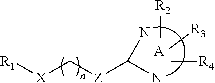
wherein
A is a ring structure which is substituted by R₂, R₃ and R₄;
X is present or absent and when present is NH, O, ester or N-monosubstituted amide;
Z is S, O, or N;
n is an integer from 0-5;
R₁ is alkyl, aryl, heteroalkyl, or heteroaryl, each with or without substitution; and
R₂, R₃, and R₄ is each present or absent, and is each independently H, alkyl, aryl, heteroalkyl, heteroaryl, each with or without substitution,
or a pharmaceutically acceptable salt thereof.

2. The method of claim 1, wherein in the compound the ring structure A is an aromatic or non-aromatic monocycle, bicycle, mono-heterocycle, or bi-heterocycle, each with or without substitution,
or a pharmaceutically acceptable salt thereof.

3. The method of claim 1, wherein R₁ is an aromatic or non-aromatic monocycle, bicycle, mono-heterocycle, or bi-heterocycle, each with or without substitution,
or a pharmaceutically acceptable salt thereof.

4. The method of claim 1, wherein at least one of ring structure A or R₁ is phenyl, pyrimidine, pyridine, imidazole, triazine, triazole, pyrimidinone, triazolotriazine, or benzimidazole, each with or without substitution,
or a pharmaceutically acceptable salt thereof.

5. The method of claim 1, wherein the ring structure A is phenyl, pyrimidine, pyridine, imidazole, triazine, triazole, pyrimidinone, or triazolotriazine, each with or without substitution,
or a pharmaceutically acceptable salt thereof.

6. The method of claim 1, wherein in the compound the ring structure A is 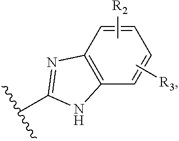
or a pharmaceutically acceptable salt thereof.

7. The method of claim 1, wherein in the compound the ring structure A is 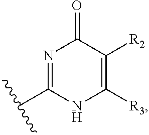
or a pharmaceutically acceptable salt thereof.

8. The method of claim 1, wherein in the compound the ring structure A is 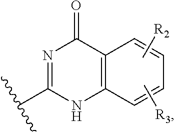
or a pharmaceutically acceptable salt thereof.

9. The method of claim 1, wherein in the compound the ring structure A is 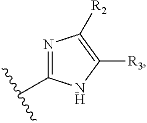
or a pharmaceutically acceptable salt thereof.

10. The method of claim 1, wherein in the compound the ring structure A is 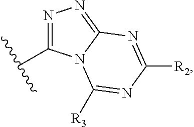
or a pharmaceutically acceptable salt thereof.

11. The method of claim 1, wherein R₄ is absent, and R₂ and R₃ are each independently H, alkyl, heteroalkyl, aryl or heteroaryl, each with or without substitution.
or a pharmaceutically acceptable salt thereof.

12. The method of claim 1, wherein X is present and X is 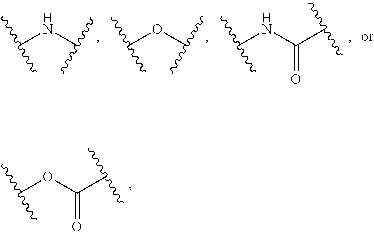
or a pharmaceutically acceptable salt thereof.

13. The method of claim 1, wherein n=1, 2, or 3;
X is present and X is 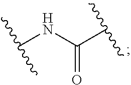
and
R₁ is unsubstituted phenyl, monosubstituted phenyl, disubstituted phenyl or trisubstituted phenyl,
or a pharmaceutically acceptable salt thereof.

14. The method of claim 13, wherein R₁ is 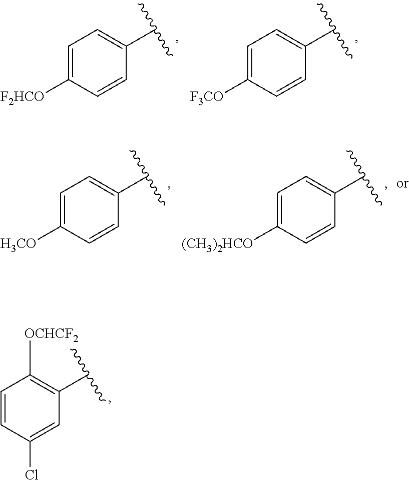
or a pharmaceutically acceptable salt thereof.

15. The method of claim 1, wherein X is absent; n=1, 2, or 3; and
R₁ is unsubstituted phenyl, monosubstituted phenyl, disubstituted phenyl, trisubstituted phenyl, or pyrimidinone, with or without substitution, fused or unfused.
or a pharmaceutically acceptable salt thereof.

16. The method of claim 15, wherein R₁ is 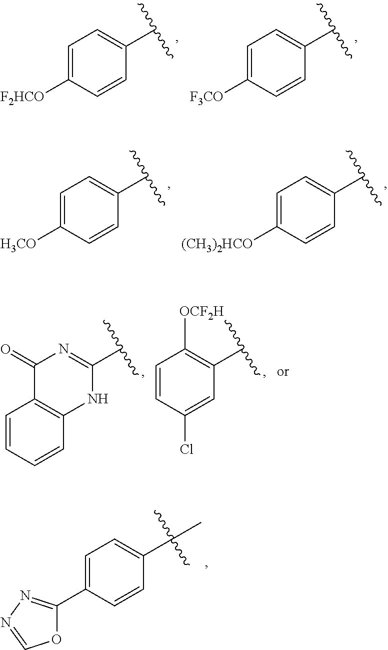
or a pharmaceutically acceptable salt thereof.

17. The method of claim 15, wherein R₁ is 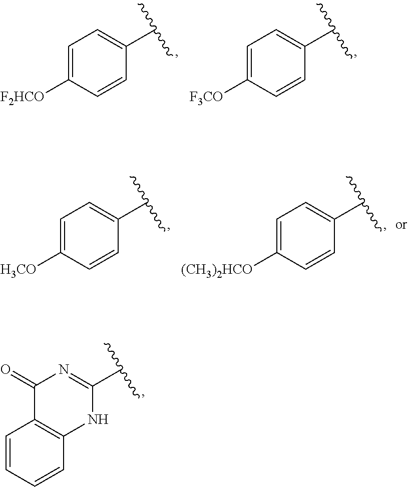
or a pharmaceutically acceptable salt thereof.

18. The method of claim 1, wherein the structure is 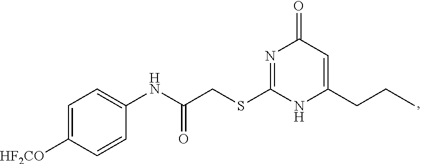
or a pharmaceutically acceptable salt thereof.

19. The method of claim 1, wherein the structure is 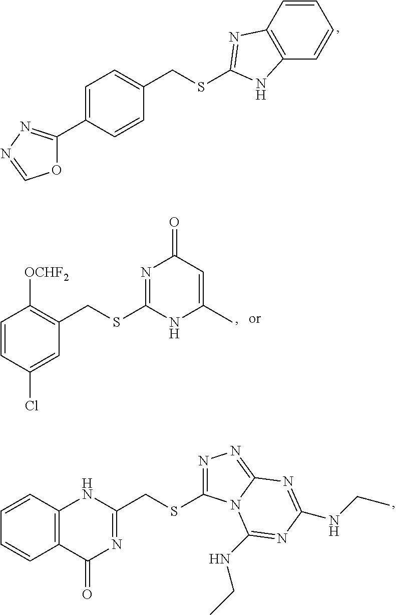
or a pharmaceutically acceptable salt thereof.

20.-21. (canceled)

22. A method for reducing one or more symptoms of disease in a mammal, comprising administering to the mammal a small molecule compound of the structure 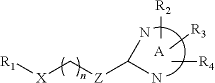
wherein
A is a ring structure which is substituted by R₂, R₃ and R₄;
X is present or absent and when present is NH, O, ester or N-monosubstituted amide;
Z is S, O, or N;
n is an integer from 0-5;
R₁ is alkyl, aryl, heteroalkyl, or heteroaryl, each with or without substitution; and
R₂, R₃, and R₄ is each present or absent, and is each independently H, alkyl, aryl, heteroalkyl, heteroaryl, each with or without substitution,
or a pharmaceutically acceptable salt thereof.

23.-29. (canceled)
